(19) United States
(12) Patent Application Publication
(10) Pub. No.: US 2023/0233546 A1
(43) Pub. Date: Jul. 27, 2023

(54) METHODS OF TREATING CANCER

(71) Applicant: Kymera Therapeutics, Inc., Watertown, MA (US)

(72) Inventors: Stefanie SCHALM, Watertown, MA (US); Dapeng Chen, Watertown, MA (US); William Proctor, Watertown, MA (US); Joseph Growney, Watertown, MA (US); Juliet Williams, Watertown, MA (US); Brad Enerson, Watertown, MA (US); Yogesh Chutake, Watertown, MA (US); Michele Mayo, Watertown, MA (US); Jianfeng Qi, Watertown, MA (US); Chris Ho, Watertown, MA (US); Matthew M. Weiss, Boston, MA (US); Haojing Rong, Watertown, MA (US)

(21) Appl. No.: 18/066,886

(22) Filed: Dec. 15, 2022

Related U.S. Application Data
(60) Provisional application No. 63/384,043, filed on Nov. 16, 2022.
(60) Provisional application No. 63/375,820, filed on Sep. 15, 2022.
(60) Provisional application No. 63/265,474, filed on Dec. 15, 2021.

(51) Int. Cl. A61K 31/4545; A61P 35/02; A61P 35/00

(52) U.S. Cl. CPC A61K 31/4545; A61P 35/02; A61P 35/00

(57) ABSTRACT

The present invention relates to methods of treating solid cancers and hematological malignances using MDM2 degraders.

### CROSS-REFERENCE TO RELATED APPLICATIONS

This application claims the benefit of priority to U.S. Provisional Appl. No. 63/265,474, filed Dec. 15, 2021, U.S. Provisional Appl. No. 63/375,820, filed Sep. 15, 2022, and U.S. Provisional Appl. No. 63/384,043, filed Nov. 16, 2022, the entirety of each of which is herein incorporated by reference.

### TECHNICAL FIELD OF THE INVENTION

The present invention relates to methods of treating solid cancers and hematological malignances using MDM2 degraders.

### BACKGROUND OF THE INVENTION

The murine double minute 2 (MDM2) oncoprotein is a key E3 ubiquitin ligase that degrades the tumor-suppressor p53. Reversible small molecule inhibitors (SMIs) of the MDM2/p53 interaction have been developed to stabilize p53 and to induce apoptosis in wildtype p53 tumors. However, MDM2 SMIs induce a p53/MDM2 feedback loop, resulting in upregulation of MDM2 protein levels and p53 pathway inhibition thus drastically limiting their biological activity and clinical application. MDM2 targeted protein degradation suppresses p53-dependent MDM2 protein feedback upregulation and is therefore expected to lead to a superior response compared to MDM2 SMIs.

A need exists to develop dosing and schedules for MDM2 degraders to improve upon the efficacy of MDM2 SMIs and other therapies and provide single-agent activity in solid cancers and hematological malignances.

### SUMMARY OF THE INVENTION

It has been found that certain MDM2 degraders are suitable for enteral and parental administration in a patient for treating solid cancers and hematological malignances. Accordingly, in one aspect, the present invention provides a method of treating a solid cancer or hematological malignancy in a patient in need thereof, comprising administering a therapeutically effective amount of an MDM2 degrader or a pharmaceutically acceptable salt thereof to the patient, wherein the MDM2 degrader is (3′R,4'S,5′R)-6″-chloro-4′-(3-chloro-2-fluorophenyl)-N-((1R,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide (Compound A), (3′R,4'S,5′R)-6″-chloro-4′-(2-chloro-3-fluoropyridin-4-yl)-N-((6S)-6-((5-(1-(2,6-dioxopiperidin-3-yl)-3-methyl-2-oxo-2,3-dihydro-1H-benzo[d]imidazol-5-yl)pentyl)carbamoyl)tetrahydro-2H-pyran-3-yl)-4,4-dimethyl-2″-oxodispiro[cyclohexane-1,2′-pyrrolidine-3′,3″-indoline]-5′-carboxamide (Compound B), (3′R,4'S,5′R)-6″-chloro-4′-(3-chloro-2-fluorophenyl)-N-((1r,4R)-4-((2-(2-(1-(2,6-dioxopiperidin-3-yl)-3-methyl-2-oxo-2,3-dihydro-1H-benzo[d]imidazol-5-yl)ethyl)-2,7-diazaspiro [3.5]nonan-7-yl)methyl)cyclohexyl)-1′-methyl-2″-oxodispiro[cyclohexane-1,2′-pyrrolidine-3′,3″-indoline]-5′-carboxamide (Compound C), (3′R,4'S,5′R)-6″-chloro-4′-(3-chloro-2-fluorophenyl)-N-(4-(4-(1-(2,6-dioxopiperidin-3-yl)-3-methyl-2-oxo-2,3-dihydro-1H-benzol[d]imidazol-5-yl)piperidine-1-carbonyl)phenyl)-2″-oxodispiro[cyclohexane-1,2′-pyrrolidine-3′,3″-indoline]-5′-carboxamide (Compound D), or (3′R,4'S,5′R)-6″-chloro-4′-(3-chloro-2-fluorophenyl)-N-((1r,4R)-4-(4-((2-(2,6-dioxopiperidin-3-yl)-1-oxoisoindolin-4-yl)ethynyl)piperidine-1-carbonyl)cyclohexyl)-2″-oxodispiro[cyclohexane-1,2′-pyrrolidine-3′,3″-indoline]-5′-carboxamide (Compound E).

In one aspect, the MDM2 degrader or a pharmaceutically acceptable salt thereof is administered at a dose of up to about 0.5 mg/kg, up to about 0.65 mg/kg, or up to about 0.8 mg/kg to the patient. In some instances, the MDM2 degrader or a pharmaceutically acceptable salt thereof is administered at a dose of from about 0.3 mg/kg to about 0.6 mg/kg or about 0.5 mg/kg to about 0.8 mg/kg. In another aspect, the MDM2 degrader or a pharmaceutically acceptable salt thereof is administered at a dose of up to about 35 mg, up to about 40 mg, up to about 50 mg, or up to about 100 mg to the patient. In some instances, the MDM2 degrader or a pharmaceutically acceptable salt thereof is administered at a dose of from about 10 mg to about 100 mg, 10 mg to about 40 mg, or from about 20 mg to about 50 mg. In a further aspect, the MDM2 degrader or a pharmaceutically acceptable salt thereof is administered at a dose of up to 5 mg/m², up to 15 mg/m², or up to 30 mg/m² to the patient. In some instances, the MDM2 degrader or a pharmaceutically acceptable salt thereof is administered at a dose of from about 1 mg/m² to about 10 mg/m² or from about 5 mg/m² to about 15 mg/m².

In one aspect, the MDM2 degrader or a pharmaceutically acceptable salt thereof is administered orally to the patient. The oral administration of the MDM2 degrader to the patient can include the MDM2 degrader in solutions, suspensions, emulsions, tablets, pills, capsules, powders, or sustained-release formulations. In other aspect, the MDM2 degrader or a pharmaceutically acceptable salt thereof is administered intravenously to the patient. The intravenous administration of the MDM2 degrader to the patient can include the MDM2 degrader in sterile injectable solutions.

In one aspect, the MDM2 degrader or a pharmaceutically acceptable salt thereof is administered to the patient once weekly (QW) or once every two weeks (Q2W) or once every three weeks (Q3W).

Also provided herein, is a pharmaceutical composition comprising the MDM2 degrader or a pharmaceutically acceptable salt thereof, and one or more pharmaceutically acceptable excipient or carrier. In some aspects, the one or more pharmaceutically acceptable excipient or carrier includes one or more diluents, preservatives, binders, lubricants, disintegrators, swelling agents, fillers, or stabilizers. In other aspects, the one or more pharmaceutically acceptable excipient or carrier includes one or more buffers, surfactants, dispersants, emulsifiers, or viscosity modifying agents.

In further aspects, the a solid cancer or hematological malignancy is selected from acute lymphoblastic leukemia (ALL), chronic lymphocytic leukemia (CLL), large granular lymphocytic leukemia (LGL-L), B-cell prolymphocytic leukemia, acute myeloid leukemia (AML), Burkitt lymphoma/leukemia, primary effusion lymphoma, peripheral T-cell lymphoma (PTCL), cutaneous T-cell lymphoma (CTCL), diffuse large B-cell lymphoma (DLBCL), advanced B-cell diffuse large B-cell lymphoma (ABC DLBCL), intravascular large B-cell lymphoma, lymphoplasmacytic lymphoma, Waldenström's macroglobulinemia (WM), splenic marginal zone lymphoma, multiple myeloma, plasmacytoma, uveal melanoma, or myelodysplastic syndrome (MDS). In some embodiments, the patient receiving the MDM2 degrader or a pharmaceutically acceptable salt thereof to treat a solid cancer or hematological malignancy has received at least one prior therapy. In some embodiments, the solid cancer or hematological malignancy is refractory. In some embodiments, the patient is a human.

These and other aspects of this disclosure will be apparent upon reference to the following detailed description. To this end, various references are set forth herein which describe in more detail certain background information and procedures and are each hereby incorporated by reference in their entirety.

### BRIEF DESCRIPTION OF THE DRAWINGS

FIG. 1 shows that Compound A, unlike SMI's such as DS-3032, degrades MDM2 at 0.4 nm.

FIG. 2 shows that Compound A, unlike SMI's such as DS-3032, strongly stabilizes p53.

FIG. 3 shows that Compound A is superior to SMI's such as DS-3032 for killing cancer cells.

FIG. 4 shows that short term exposure to Compound A, unlike SMI's such as DS-3032, is sufficient to commit cells to undergo apoptosis.

FIG. 5 shows a schematic of the washout experiments performed in RS4;11 cells.

FIG. 6 shows that a single dose of Compound A achieves sustained tumor regression in a RS4;11 mouse xenograft model (A) and MDM2 degradation at 1 mg/kg Compound A leads to a dose dependent increase in p53, p21, and PUMA.

FIGS. 7A and 7B shows that Compound A (1 mg/kg, Q3W) achieves tumor regression in a CTG-2227 AML patient-derived xenograft (PDX) model and partial responses in CTG-2240 and CTG-2700 AML PDX models.

FIGS. 8A and 8B shows the combinatorial benefit of Compound A with venetoclax and midostaurin in MOLM-13 cell line.

FIG. 9 shows the significant combinatorial benefit of Compound A with standard of care in AML in vivo model.

FIG. 10 shows that Compound A is active across multiple heme indications in vitro with AML, T cell lymphomas, mantle cell lymphoma, and DLBCL being the most sensitive.

FIG. 11 shows that Compound A is highly active in p53^{WT} ABC-subtype DLBCL. Compound A was highly active in OCI-LY10 p53^{WT} ABC-subtype DLBCL xenograftmodel (A) but not TMD8 p53^{MUT} ABC-subtype DLBCL xenograft model (B).

### DETAILED DESCRIPTION OF CERTAIN EMBODIMENTS

### 1. General Description of Certain Embodiments of the Invention

The MDM2 degraders provided herein are highly potent heterobifunctional small molecule therapeutic agents targeting MDM2 to mediate the selective degradation of MDM2 protein. The provided MDM2 degraders display superior activity compared to SMIs of MDM2 in wildtype p53 cell lines and xenograft models. For instance in acute lymphoblastic leukemia (ALL) cell line RS4;11, Compound A can overcome the p53-dependent upregulation of MDM2 protein levels as seen for reversible SMIs. Short 2 hour exposures of Compound A can more potently stabilize p53 than SMIs. In addition, washout experiments in these cells showed that a pulsed dose of Compound A can lead to apoptosis mediated through p53 target genes. The superior MDM2/p53 pathway inhibition and induction of apoptosis by Compound A translates into a >200-fold stronger cell growth inhibition, compared to SMIs, across a panel of solid and hematological tumor cell lines. In some embodiments, provided herein is a treatment of adult patients with solid cancers or hematological malignances who have received at least one prior therapy. The MDM2 degraders of the current invention are provided by oral and intravenous administration at the doses and schedules described herein.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 2. Definitions

As used in the specification and appended claims, unless specified to the contrary, the following terms and abbreviations have the following meanings.

As used herein, the term “about” refers to within 20% of a given value. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, the term “Compound A” refers to (3′R,4'S,5′R)-6″-chloro-4′-(3-chloro-2-fluorophenyl)-N-((1R,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide having the formula:

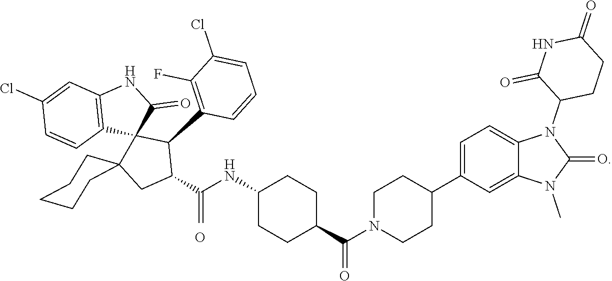 [SMILES: Cn1c(=O)n(C2CCC(=O)NC2=O)c2ccc(C3CCN(C(=O)[C@H]4CC[C@H](NC(=O)[C@@H]5CC6(CCCCC6)[C@@]6(C(=O)Nc7cc(Cl)ccc76)[C@H]5c5cccc(Cl)c5F)CC4)CC3)cc21]

In some embodiments, Compound A or a pharmaceutically acceptable salt thereof, is in amorphous form. In some embodiments, Compound A or a pharmaceutically acceptable salt thereof, is in crystalline form.

As used herein, the term “Compound B” refers to (3′R,4'S,5′R)-6″-chloro-4′-(2-chloro-3-fluoropyridin-4-yl)-N-((6S)-6-((5-(1-(2,6-dioxopiperidin-3-yl)-3-methyl-2-oxo-2,3-dihydro-1H-benzo[d]imidazol-5-yl)pentyl)carbamoyl)tetrahydro-2H-pyran-3-yl)-4,4-dimethyl-2″-oxodispiro[cyclohexane-1,2′-pyrrolidine-3′,3″-indoline]-5′-carboxamide having the formula:

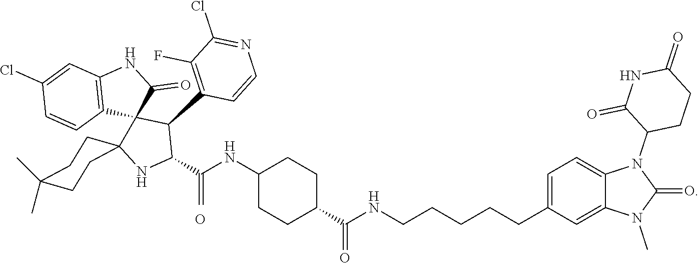

In some embodiments, Compound B or a pharmaceutically acceptable salt thereof, is in amorphous form. In some embodiments, Compound B or a pharmaceutically acceptable salt thereof, is in crystalline form.

As used herein, the term “Compound C” refers to (3′R,4'S,5′R)-6″-chloro-4′-(3-chloro-2-fluorophenyl)-N-((1r,4R)-4-((2-(2-(1-(2,6-dioxopiperidin-3-yl)-3-methyl-2-oxo-2,3-dihydro-1H-benzo[d]imidazol-5-yl)ethyl)-2,7-diazaspiro[3.5]nonan-7-yl)methyl)cyclohexyl)-1′-methyl-2″-oxodispiro[cyclohexane-1,2′-pyrrolidine-3′,3″-indoline]-5′-carboxamide having the formula:

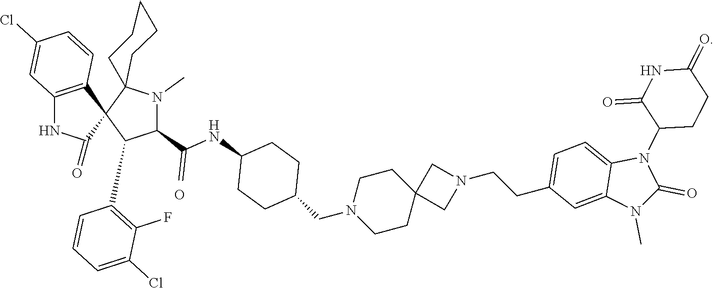 [SMILES: CN1[C@@H](C(=O)N[C@H]2CC[C@H](CN3CCC4(CC3)CN(CCc3ccc5c(c3)n(C)c(=O)n5C3CCC(=O)NC3=O)C4)CC2)[C@H](c2cccc(Cl)c2F)[C@]2(C(=O)Nc3cc(Cl)ccc32)C12CCCCC2]

In some embodiments, Compound C or a pharmaceutically acceptable salt thereof, is in amorphous form. In some embodiments, Compound C or a pharmaceutically acceptable salt thereof, is in crystalline form.

As used herein, the term “Compound D” refers to (3′R,4'S,5′R)-6″-chloro-4′-(3-chloro-2-fluorophenyl)-N-(4-(4-(1-(2,6-dioxopiperidin-3-yl)-3-methyl-2-oxo-2,3-dihydro-1H-benzo[d]imidazol-5-yl)piperidine-1-carbonyl)phenyl)-2″-oxodispiro[cyclohexane-1,2′-pyrrolidine-3′,3″-indoline]-5′-carboxamide having the formula:

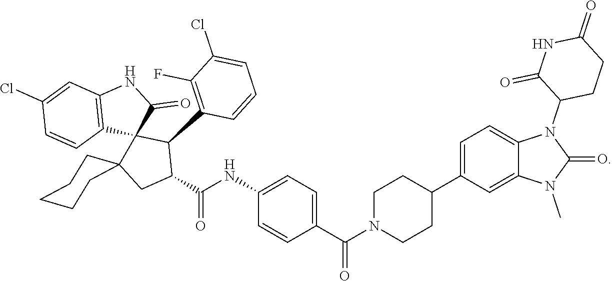

In some embodiments, Compound D or a pharmaceutically acceptable salt thereof, is in amorphous form. In some embodiments, Compound D or a pharmaceutically acceptable salt thereof, is in crystalline form.

As used herein, the term “Compound E” refers to (3′R,4'S,5′R)-6″-chloro-4′-(3-chloro-2-fluorophenyl)-N-((1r,4R)-4-(4-((2-(2,6-dioxopiperidin-3-yl)-1-oxoisoindolin-4-yl)ethynyl)piperidine-1-carbonyl)cyclohexyl)-2″-oxodispiro[cyclohexane-1,2′-pyrrolidine-3′,3″-indoline]-5′-carboxamide having the formula:

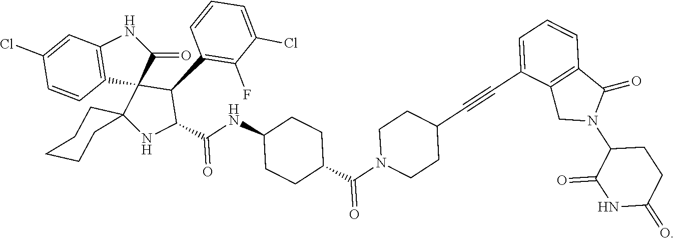 [SMILES: O=C1CCC(N2Cc3c(C#CC4CCN(C(=O)[C@H]5CC[C@H](NC(=O)[C@@H]6NC7(CCCCC7)[C@@]7(C(=O)Nc8cc(Cl)ccc87)[C@H]6c6cccc(Cl)c6F)CC5)CC4)cccc3C2=O)C(=O)N1]

In some embodiments, Compound E or a pharmaceutically acceptable salt thereof, is in amorphous form. In some embodiments, Compound E or a pharmaceutically acceptable salt thereof, is in crystalline form.

As used herein, the term “inhibitor” is defined as a compound that binds to and/or inhibits MDM2 protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “MDM2 degrader” refers to an agent that degrades MDM2 protein. Various MDM2 degraders have been described previously, for example, in WO 2021/188948, the contents of which are incorporated herein by reference in their entireties. In certain embodiments, an MDM2 degrader has an DC₅₀ of less than about 50 μM, less than about 1 μM, less than about 500 nM, less than about 100 nM, less than about 10 nM, or less than about 1 nM. In certain embodiments, the MDM2 degrader is Compound A, B, C, D, or E disclosed herein.

The term “patient,” as used herein, means an animal, preferably a mammal, and most preferably a human.

As used herein, the term “mg/kg” or “mpk” refers to the milligram of medication (for example, Compound A) per kilogram of the body weight of the subject taking the medication.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

The term “pharmaceutically acceptable excipient or carrier” refers to a non-toxic excipient or carrier that does not destroy the pharmacological activity of the compound with which it is formulated. Pharmaceutically acceptable excipient or carrier that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “therapeutically effective amount” as used herein refers to an amount of MDM2 degrader that is sufficient to treat the stated disease, disorder, or condition or have the desired stated effect on the disease, disorder, or condition or one or more mechanisms underlying the disease, disorder, or condition in a subject. In certain embodiments, when Compound A is administered for the treatment of a solid cancer or hematological malignancy, therapeutically effective amount refers an amount of Compound A which, upon administration to a subject, treats or ameliorates the solid cancer or hematological malignancy in the subject, or exhibits a detectable therapeutic effect in the subject that results in partial to complete tumor regression.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

### 3. Description of Exemplary Embodiments

According to one aspect, the invention provides a method for treating a solid cancer or hematological malignancy in a patient in need thereof, comprising administering a therapeutically effective amount of an MDM2 degrader (e.g., Compound A, B, C, D, or E), or a pharmaceutically acceptable salt thereof. In some embodiments, the method comprises administering up to 50 mg of an MDM2 degrader (e.g., Compound A, B, C, D, or E), or a pharmaceutically acceptable salt thereof in a single or divided dose.

Pharmaceutically Acceptable Compositions

According to one embodiment, the invention provides a composition comprising an MDM2 degrader of this invention (e.g., Compound A, B, C, D, or E) or a pharmaceutically acceptable derivative thereof and a pharmaceutically acceptable excipient or carrier. The amount of MDM2 degrader in compositions of this invention is such that it is effective to measurably degrade and/or inhibit MDM2 protein, or a mutant thereof, in a patient. In certain embodiments, a composition of this invention is formulated for administration to a patient in need of such composition. In some embodiments, a composition of this invention is formulated for oral administration to a patient. In some embodiments, a composition of this invention is formulated for intravenous administration to a patient.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular MDM2 degrader in the composition.

Compositions

The dosage forms disclosed herein include pharmaceutically acceptable salts of an MDM2 degraders (e.g., Compound A, B, C, D, or E). In some embodiments, the dosage forms can be formulated for enteral or parenteral administration. The MDM2 degrader can be combined with one or more pharmaceutically acceptable carriers that are considered safe and effective to form a unit dosage as described herein, and may be administered to an individual without causing undesirable biological side effects or unwanted interactions.

These dosage forms can take the form of solutions, suspensions, emulsion, tablets, pills, capsules, powders, sustained-release formulations and the like.

In one preferred embodiment, the dosage form is in the form of a solution, including an MDM2 degraders (e.g., Compound A, B, C, D, or E). The dosage form is administered to the subject in need thereof, for a time period effective to ameliorate the patient condition (e.g., a solid cancer or hematological malignancy).

Excipients and Carriers

Pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions.

Suitable pharmaceutical excipients include starch, glucose, sucrose, gelatin, lactose, malt, rice, flour, chalk, silica gel, sodium stearate, glycerol monostearate, talc, sodium chloride, glycerol, propylene, glycol, water, ethanol and the like. The pharmaceutical composition may also contain wetting or emulsifying agents or suspending/diluting agents, or pH buffering agents, or agents for modifying or maintaining the rate of release of the disclosed salts, all of which are disclosed further herein.

Administration and Dosage

As described herein, the MDM2 degraders provided herein are administered by parenteral and enteral routes. In some embodiments, an MDM2 degrader (e.g., Compound A, B, C, D, or E) or a pharmaceutically acceptable salt thereof, is administered intravenously. In some embodiments, an MDM2 degrader (e.g., Compound A, B, C, D, or E) or a pharmaceutically acceptable salt thereof is administered by an IV injection. In some embodiments, an MDM2 degrader (e.g., Compound A, B, C, D, or E) or a pharmaceutically acceptable salt thereof is administered by an IV infusion.

As described herein, an MDM2 degrader (e.g., Compound A, B, C, D, or E) or a pharmaceutically acceptable salt thereof, is administered enterally. In some embodiments, an MDM2 degraders (e.g., Compound A, B, C, D, or E) or a pharmaceutically acceptable salt thereof is administered in amorphous form (e.g., pressed into pills or in capsules). In some embodiments, an MDM2 degrader (e.g., Compound A, B, C, D, or E) or a pharmaceutically acceptable salt thereof is administered as a lyophilized powder.

In some embodiments, a method of the invention comprises orally administering to a patient a pharmaceutical composition comprising an MDM2 degrader. In some embodiments, a pharmaceutical composition is a solid pharmaceutical composition. In some embodiments, the solid pharmaceutical composition is a powder. In some embodiments, the pharmaceutical composition is lyophilized powder. In some embodiments, the solid pharmaceutical composition is granules. In some embodiments, the solid pharmaceutical composition of the invention is tablets. In some embodiments, the solid pharmaceutical composition is capsules. In some embodiments, the solid pharmaceutical composition is pills. In some embodiments, the solid pharmaceutical composition is suspensions. In some embodiments, the solid pharmaceutical composition is emulsions. In some embodiments, the solid pharmaceutical composition is solutions.

In some embodiments, the methods and uses described herein, such as the method of or use in treating a solid cancer or hematological malignancy in a patient in need thereof, is achieved by administering (e.g., orally or intravenously) a therapeutically effective amount of an MDM2 degrader (e.g., Compound A, B, C, D, or E), such as up to 100 mg in a single or multiple dosage units. In some embodiments, the method can include administering (e.g., orally or intravenously), in a single or multiple dosage units ranging from about 1 to about 100 mg/dosage form, such as about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, or about 100 mg. For example, an enteric tablet or liquid formulation can include 5 mg, 10 mg, 15 mg, 20 mg, 25 mg, 30 mg, 35 mg, 40 mg, 45 mg, or 50 mg/dosage form of an MDM2 degrader (e.g., Compound A, B, C, D, or E) or a pharmaceutically acceptable salt thereof.

In some embodiments, the MDM2 degrader (e.g., Compound A, B, C, D, or E) or a pharmaceutically acceptable salt thereof is intravenously administered at a dose of up to 5 mg to the patient. In some embodiments, the MDM2 degrader (e.g., Compound A, B, C, D, or E) or a pharmaceutically acceptable salt thereof is intravenously administered at a dose of up to 10 mg to the patient. In some embodiments, the MDM2 degrader (e.g., Compound A, B, C, D, or E) or a pharmaceutically acceptable salt thereof is intravenously administered at a dose of up to 15 mg to the patient. In some embodiments, the MDM2 degrader (e.g., Compound A, B, C, D, or E) or a pharmaceutically acceptable salt thereof is intravenously administered at a dose of up to 20 mg to the patient. In some embodiments, the MDM2 degrader (e.g., Compound A, B, C, D, or E) or a pharmaceutically acceptable salt thereof is intravenously administered at a dose of up to 25 mg to the patient. In some embodiments, the MDM2 degrader (e.g., Compound A, B, C, D, or E) or a pharmaceutically acceptable salt thereof is intravenously administered at a dose of up to 30 mg to the patient. In some embodiments, the MDM2 degrader (e.g., Compound A, B, C, D, or E) or a pharmaceutically acceptable salt thereof is intravenously administered at a dose of up to 35 mg to the patient. In some embodiments, the MDM2 degrader (e.g., Compound A, B, C, D, or E) or a pharmaceutically acceptable salt thereof is intravenously administered at a dose of up to 40 mg to the patient. In some embodiments, the MDM2 degrader (e.g., Compound A, B, C, D, or E) or a pharmaceutically acceptable salt thereof is intravenously administered at a dose of up to 45 mg to the patient. In some embodiments, the MDM2 degrader (e.g., Compound A, B, C, D, or E) or a pharmaceutically acceptable salt thereof is intravenously administered at a dose of up to 50 mg to the patient. In some embodiments, the MDM2 degrader (e.g., Compound A, B, C, D, or E) or a pharmaceutically acceptable salt thereof is intravenously administered at a dose of from about 10 mg to about 40 mg to the patient. In some embodiments, the MDM2 degrader (e.g., Compound A, B, C, D, or E) or a pharmaceutically acceptable salt thereof is intravenously administered at a dose of from about 20 mg to about 50 mg to the patient, such as about 30 mg, 35 mg, or 40 mg. In some embodiments, the MDM2 degrader (e.g., Compound A, B, C, D, or E) or a pharmaceutically acceptable salt thereof is intravenously administered at a dose of about 35 mg to the patient.

In some embodiments, a pharmaceutical composition is provided, wherein, the pharmaceutically composition comprises 5 mg to about 50 mg of the MDM2 degrader (e.g., Compound A, B, C, D, or E), or a pharmaceutically acceptable salt thereof, and one or more pharmaceutically acceptable excipient or carrier. In some embodiments, a pharmaceutical composition is provided, wherein, the pharmaceutically composition comprises 25 mg to about 45 mg of the MDM2 degrader (e.g., Compound A, B, C, D, or E), or a pharmaceutically acceptable salt thereof, and one or more pharmaceutically acceptable excipient or carrier.

In some embodiments, the MDM2 degrader (e.g., Compound A, B, C, D, or E), or a pharmaceutically acceptable salt thereof, or a pharmaceutical composition thereof, is administered to a mouse at a dose of up to about 10 mg/kg for intravenous administration. Accordingly, in some embodiments, the MDM2 degrader (e.g., Compound A, B, C, D, or E), or a pharmaceutically acceptable salt thereof, or a pharmaceutical composition thereof, is administered intravenously to a patient at a dose of up to about 30 mg/m². In some embodiments, the MDM2 degrader (e.g., Compound A, B, C, D, or E), or a pharmaceutically acceptable salt thereof, or a pharmaceutical composition thereof, is administered intravenously to a patient at a dose of up to about 25 mg/m², or up to about 20 mg/m², or up to about 15 mg/m². In some embodiments, the MDM2 degrader (e.g., Compound A, B, C, D, or E), or a pharmaceutically acceptable salt thereof, or a pharmaceutical composition thereof, is administered intravenously to a patient at a dose of about 1 mg/m² to about 5 mg/m², or about 3 mg/m² to about 8 mg/m², or about 5 mg/m² to about 10 mg/m², or about 7 mg/m² to about 12 mg/m², or about 10 mg/m² to about 15 mg/m², or about 12 mg/m² to about 7 mg/m², or about 15 mg/m² to about 20 mg/m², or about 17 mg/m² to about 22 mg/m², or about 20 mg/m² to about 25 mg/m², or about 22 mg/m² to about 27 mg/m². In some embodiments, the MDM2 degrader (e.g., Compound A, B, C, D, or E), or a pharmaceutically acceptable salt thereof, or a pharmaceutical composition thereof, is administered intravenously to a patient at a dose of about 30 mg/m², about 27 mg/m², about 20 mg/m², about 17 mg/m², about 15 mg/m², about 12 mg/m², about 10 mg/m², about 7 mg/m², about 5 mg/m², about 3 mg/m², or about 1 mg/m².

In some embodiments, the MDM2 degrader (e.g., Compound A, B, C, D, or E), or a pharmaceutically acceptable salt thereof, or a pharmaceutical composition thereof, is administered to a mouse at a dose of up to about 10 mg/kg for intravenous administration. Accordingly, in some embodiments, the MDM2 degrader (e.g., Compound A, B, C, D, or E), or a pharmaceutically acceptable salt thereof, or a pharmaceutical composition thereof, is administered orally to a patient at a dose of up to about 0.8 mg/kg. In some embodiments, the MDM2 degrader (e.g., Compound A, B, C, D, or E), or a pharmaceutically acceptable salt thereof, or a pharmaceutical composition thereof, is administered orally to a patient at a dose of up to about 0.6 mg/kg, or up to about 0.3 mg/kg, or up to about 0.1 mg/kg. In some embodiments, the MDM2 degrader (e.g., Compound A, B, C, D, or E), or a pharmaceutically acceptable salt thereof, or a pharmaceutical composition thereof, is administered orally to a patient at a dose of about 0.01 mg/kg to about 0.05 mg/kg, or about 0.03 mg/kg to about 0.08 mg/kg, or about 0.05 mg/kg to about 0.1 mg/kg, or about 0.07 mg/kg to about 0.12 mg/kg, or about 0.1 mg/kg to about 0.15 mg/kg, or about 0.12 mg/kg to about 0.17 mg/kg. In some embodiments, the MDM2 degrader (e.g., Compound A, B, C, D, or E), or a pharmaceutically acceptable salt thereof, or a pharmaceutical composition thereof, is administered orally to a patient at a dose of about 1 mg/kg, about 0.8 mg/kg, about 0.5 mg/kg, about 0.3 mg/kg, about 0.1 mg/kg, about 0.08 mg/kg, or about 0.06 mg/kg.

Dosing Schedule

As provided in view of preclinical data described herein, an MDM2 degrader (e.g., Compound A, B, C, D, or E) or a pharmaceutically acceptable salt thereof, or a pharmaceutical composition thereof, is administered to a patient at a dosing schedule appropriate to give the desired tumor regression effect with minimum side effects. In some embodiments, the MDM2 degrader (e.g., Compound A, B, C, D, or E) or pharmaceutical composition thereof is administered to a patient once every 1, 2, 3, 4, 5, 6, 7, 14, or 21 days. In some embodiments, the MDM2 degrader (e.g., Compound A, B, C, D, or E) or pharmaceutical composition thereof is administered to a patient daily (QD). In some embodiments, the MDM2 degrader (e.g., Compound A, B, C, D, or E) or pharmaceutical composition thereof is administered to a patient biweekly (BW). In some embodiments, the MDM2 degrader (e.g., Compound A, B, C, D, or E) or pharmaceutical composition thereof is administered to a patient weekly (QW). In some embodiments, the MDM2 degrader (e.g., Compound A, B, C, D, or E) or pharmaceutical composition thereof is administered to a patient every two weeks (Q2W). In some embodiments, the MDM2 degrader (e.g., Compound A, B, C, D, or E) or pharmaceutical composition thereof is administered to a patient every three weeks (Q3W).

In some embodiments, an IV infusion of a pharmaceutical composition of the invention lasts about 5-30 minutes. In some embodiments, an IV infusion of a pharmaceutical composition of the invention lasts about 30-90 minutes. In some embodiments, an IV infusion of a pharmaceutical composition of the invention lasts about 5, 10, 15, 20, 25, 30, 35, 40, 45, 50, 55, 60, 65, 70, 75, 80, 85, or 90 minutes. In some embodiments, an IV infusion of a pharmaceutical composition of the invention lasts about 2, 2.5, 3, 3.5, or 4 hours.

In some embodiments, a pharmaceutical composition of the invention is administered intravenously weekly at a dose of from about 0.1 mg/m² to about 30 mg/m². In some embodiments, a pharmaceutical composition of the invention is administered weekly at a dose of from about 1 mg/m² to about 10 mg/m².

Formulation of Pharmaceutical Compositions

The administration of the MDM2 degraders of the present invention may be by any suitable means that results in a concentration of the drug that, combined with the other component, is able to ameliorate the patient condition (e.g., a solid cancer or hematological malignancy).

While it is possible for the active ingredients of the combination to be administered as the pure chemical, it is preferable to present them as a pharmaceutical composition, also referred to in this context as pharmaceutical formulation. Possible compositions include those suitable for oral, rectal, topical (including transdermal, buccal and sublingual), or parenteral (including subcutaneous, intramuscular, intravenous and intradermal) administration.

More commonly these pharmaceutical formulations are prescribed to the patient in “patient packs” containing a number dosing units or other means for administration of metered unit doses for use during a distinct treatment period in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions. Thus, the invention further includes a pharmaceutical formulation, as herein before described, in combination with packaging material suitable for said formulations. In such a patient pack the intended use of a formulation for the combination treatment can be inferred by instructions, facilities, provisions, adaptations and/or other means to help using the formulation most suitably for the treatment. Such measures make a patient pack specifically suitable and adapted for use for treatment with the combination of the present invention.

The drug may be contained in any appropriate amount in any suitable carrier substance, and may be present in an amount of 1-99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active drug substantially immediately upon administration or at any predetermined time or time period after administration.

The controlled release formulations include (i) formulations that create a substantially constant concentration of the drug within the body over an extended period of time; (ii) formulations that after a predetermined lag time create a substantially constant concentration of the drug within the body over an extended period of time; (iii) formulations that sustain drug action during a predetermined time period by maintaining a relatively, constant, effective drug level in the body with concomitant minimization of undesirable side effects associated with fluctuations in the plasma level of the active drug substance; (iv) formulations that localize drug action by, e.g., spatial placement of a controlled release composition adjacent to or in the diseased tissue or organ; and (v) formulations that target drug action by using carriers or chemical derivatives to deliver the drug to a particular target cell type.

Administration of drugs in the form of a controlled release formulation is especially preferred in cases in which the drug in combination, has (i) a narrow therapeutic index (i.e., the difference between the plasma concentration leading to harmful side effects or toxic reactions and the plasma concentration leading to a therapeutic effect is small; in general, the therapeutic index, TI, is defined as the ratio of median lethal dose (LD₅₀) to median effective dose (ED₅₀)); (ii) a narrow absorption window in the gastro-intestinal tract; or (iii) a very short biological half-life so that frequent dosing during a day is required in order to sustain the plasma level at a therapeutic level.

Any of a number of strategies can be pursued in order to obtain controlled release in which the rate of release outweighs the rate of metabolism of the drug in question. Controlled release may be obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner (single or multiple unit tablet or capsule compositions, oil solutions, suspensions, emulsions, microcapsules, microspheres, nanoparticles, patches, and liposomes).

Parenteral Compositions

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The components of the composition are can be either separately or mixed together in unit dosage form, for example, as a dry lyophilized powder (which can be reconstituted before use with a carrier such as saline) or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to buffers, surfactants, dispersants, emulsifiers, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-laurylβiminodipropionate, myristoamphoacetate, lauryl betaine, and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

In some embodiments, the parental compositions (e.g, for intravenous administration) are packaged in solutions with sterile isotonic aqueous buffers. In some embodiments, the parental compositions are buffered to a pH of 3-8 for parenteral administration upon reconstitution. Suitable buffers include, but are not limited to, phosphate buffers, acetate buffers, and citrate buffers.

In some embodiments, a buffering agent is at an amount to adjust pH of a pharmaceutical composition of the invention to about 3-8. In some embodiments, a buffering agent is added at an amount of about 0.1-5 mg per mg of MDM2 degrader (e.g., Compound A, B, C, D, or E), or a pharmaceutically acceptable thereof.

In some embodiments, a liquid pharmaceutical composition of the invention is at a pH of about 3-8.

Water-soluble polymers are often used in formulations for parenteral administration. Suitable water-soluble polymers include, but are not limited to, polyvinylpyrrolidone, dextran, carboxymethylcellulose, and polyethylene glycol.

In some embodiments, the parental composition may include a solubilizing agent. In some embodiments, the solubilizing agent is a cyclodextrin. Cyclodextrines include members of a family of cyclic oligosaccharides, composed of 5 or more α-D-glucopyranoside units linked between positions 1 and 4, as known for amylose, a fragment of starch. In some embodiments, the cyclodextrin is an α-cyclodextrin, β-cyclodextrin, and/or γ-cyclodextrin, In certain embodiments, the cyclodextrin is β-cyclodextrins, such as hydroxypropyl-β-cyclodextrin (HP-β-CD). In some embodiments, the incorporation of a β-cyclodextrin (e.g., HPβCD) in the intravenous compositions of the present invention improve the dissolution of a MDM2 degrader (e.g., Compound A, B, C, D, or E) to provide a clear homogeneous solution for injection. In some embodiments the amount of a cyclodextrin (e.g., e.g., HPβCD) added to the parental composition of the present invention (e.g, for intravenous administration) may include from about 1% to about 50% w/w or w/v, such as about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49% w/w or w/v. In certain embodiments, the amount of cyclodextrin (e.g., e.g., HPβCD) is about 20% w/w or w/v.

In some embodiments, the MDM2 degrader (e.g., Compound A, B, C, D, or E), or a pharmaceutically acceptable salt thereof, is added to the parental composition of the present invention (e.g, for intravenous administration) in an amount of from about 0.01 to about 0.5 mg/mL, such as about 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, or 0.49 mg/mL. In certain embodiments, the MDM2 degrader (e.g., Compound A, B, C, D, or E), or a pharmaceutically acceptable salt thereof, is added to the the parental composition up to about 0.1 mg/mL.

Sterile injectable solutions of the present invention can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In some embodiments, the parenteral formulations described herein can be formulated for controlled release including immediate release, delayed release, extended release, pulsatile release, and combinations thereof.

In some embodiments, the invention provides a liquid pharmaceutical composition prepared by dissolving a solid pharmaceutical composition of the invention in water. In some embodiments, the invention provides a liquid pharmaceutical composition prepared by dissolving a solid pharmaceutical composition of the invention in an injectable medium (e.g., saline or 5% dextrose). In some embodiments, the invention provides a liquid pharmaceutical composition prepared by reconstitute a solid pharmaceutical composition of the invention in water, followed by dilution with saline or 5% dextrose. In some embodiments, a liquid pharmaceutical composition is diluted into a saline or 5% dextrose IV bag for IV administration. In some embodiments, a liquid pharmaceutical composition in a saline or 5% dextrose IV bag is stored under room temperature (about 20-25° C.) for up to about 4 hours before IV administration. In some embodiments, a liquid pharmaceutical composition in a saline or 5% dextrose IV bag is stored under refrigerated (about 2-8° C.) conditions for up to about 20 hours before IV administration. In some embodiments, a liquid pharmaceutical composition in a saline or 5% dextrose IV bag is stored under refrigerated (about 2-8° C.) conditions for up to about 20 hours, followed by storage under room temperature (about 20-25° C.) for up to about 4 hours, before IV administration.

Uses of Compounds and Pharmaceutically Acceptable Compositions

Compounds and compositions described herein are generally useful for the degradation and/or inhibition of MDM2 protein activity.

In some embodiments, the invention provides MDM2 degraders that modulate targeted ubiquitination and degradation of MDM2 protein. Provided compounds are degraders and/or inhibitors of MDM2 protein and are therefore useful for treating one or more disorders associated with activity of MDM2 protein. Thus, in certain embodiments, the present invention provides a method for treating a MDM2-mediated disorder comprising the step of administering to a patient in need thereof a compound of the present invention, or pharmaceutically acceptable composition thereof.

As used herein, the terms “MDM2-mediated” disorders, diseases, and/or conditions as used herein means any disease or other deleterious condition in which MDM2 protein or a mutant thereof, are known to play a role. Accordingly, another embodiment of the present invention relates to treating or lessening the severity of one or more diseases in which MDM2 protein or a mutant thereof, are known to play a role.

In some embodiments, the present invention provides a method for treating one or more disorders, diseases, and/or conditions wherein the disorder, disease, or condition is a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In some embodiments, the cancer is selected from adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, rhabdoid (e.g., atypical teratoid rhabdoid tumor), B-cell chronic lymphocytic leukemia, B-cell prolymphocytic leukemia, B-cell lymphoma, basal cell carcinoma, bile duct cancer, biliary tract cancer, bladder cancer, blastoma, bone cancer, myelofibrosis, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Ewing sarcoma, Schwannomatosis, seminoma, Sertoli cell tumor, sex cord-gonadal stromal tumor, signet ring cell carcinoma, skin cancer, Merkel cell carcinoma, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma thyroid cancer, transitional cell carcinoma, throat cancer, urachal cancer, urogenital cancer, urothelial carcinoma, uveal melanoma, uterine cancer, verrucous carcinoma, visual pathway ghoma, vulvar cancer, vaginal cancer, Waldenstrom's macroglobulinemia, Warthin's tumor, and Wilms' tumor.

MDM2 hyperactivity, due to amplification/overexpression or mutational inactivation of the ARF locus, inhibits the function of wild-type p53 and can lead to the development of a wide variety of cancers. In some embodiments, the MDM2 hyperactivity which can be treated according to the methods of this invention is a human cancer. In some embodiments, the human cancer which can be treated according to the methods of this invention is selected from a solid cancer or hematological malignancy. In some embodimentsm, the wild-type p53 cancer is mesothelioma, melanoma, DLBCL, prostate cancer, cholangiocarcinoma, cervical cancer, AML, renal cell cancer, uveal melanoma, thyroid cancer, liposarcoma, HCC, or breast cancer.

In some embodiments, the solid cancer includes solid tumors that have an abnormal mass of tissue that may not contain cysts or liquid areas. Solid tumors may be benign or malignant. In some embodiments, examples of solid tumors include sarcomas, carcinomas, and lymphomas. In some embodiments, the solid cancer is carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, gastrointestinal cancer, such as colon carcinoma or colorectal adenoma, a tumor of the neck and head, an epidermal hyperproliferation, prostate hyperplasia, a neoplasia, a neoplasia of epithelial character, adenoma, adenocarcinoma, keratoacanthoma, epidermoid carcinoma, large cell carcinoma, non-small-cell lung carcinoma, such as Hodgkin's and Non-Hodgkin's, a mammary carcinoma, follicular carcinoma, undifferentiated carcinoma, papillary carcinoma, seminoma, melanoma, an IL-1 driven disorder, an MyD88 driven disorder, or Smoldering of indolent multiple myeloma. In some embodiments, the solid tumor is refractory (e.g., treatment resistant). In some embodiments, the hematological malignancy is a cancer that affects the blood, bone marrow, and lymph nodes. In some embodiments the hematological malignancy includes leukemias, lymphomas, and myelomas, such as acute lymphoblastic leukemia (ALL), chronic lymphocytic leukemia (CLL), large granular lymphocytic leukemia (LGL-L), B-cell prolymphocytic leukemia, acute myeloid leukemia (AML), Burkitt lymphoma/leukemia, primary effusion lymphoma, peripheral T-cell lymphoma (PTCL), cutaneous T-cell lymphoma (CTCL), diffuse large B-cell lymphoma (DLBCL), advanced B-cell diffuse large B-cell lymphoma (ABC DLBCL), intravascular large B-cell lymphoma, lymphoplasmacytic lymphoma, Waldenström's macroglobulinemia (WM), splenic marginal zone lymphoma, multiple myeloma, plasmacytoma, uveal melanoma, or myelodysplastic syndrome (MDS). In some embodiments, the hematological malignancy is refractory (e.g., treatment resistant).

In some embodiments, the AML is caused by protein (e.g., of KMT2A or MLL) mutation or fusion. In some embodiments, the AML is a mutant or fusion protein AML, such as IDH1, DNMT3A, NPM1, ASXL1, FLT3-ITD, KMT2A-MLLT3, MLL-MLLT3, or MLL-AF9.

In some embodiment, the present disclosure provides a method of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratosis, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

In some embodiments, the cancer is a leukaemia, for example a leukaemia selected from acute monocytic leukemia, acute myelogenous leukemia, chronic myelogenous leukemia, chronic lymphocytic leukemia and mixed lineage leukaemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In some embodiments, the present invention provides a method of treating triple negative breast cancer in a patient in need thereof, comprising administering an MDM2 degrader (e.g., Compound A, B, C, D, or E) of the present invention, or a pharmaceutically acceptable salt thereof.

In some embodiments, the present invention provides a method of treating acute lymphoblastic leukemia (ALL), comprising administering an MDM2 degrader (e.g., Compound A, B, C, D, or E) of the present invention, or a pharmaceutically acceptable salt thereof.

In some embodiments, the present invention provides a method of treating chronic lymphocytic leukemia (CLL), comprising administering an MDM2 degrader (e.g., Compound A, B, C, D, or E) of the present invention, or a pharmaceutically acceptable salt thereof.

In some embodiments, the present invention provides a method of treating large granular lymphocytic leukemia (LGL-L), comprising administering an MDM2 degrader (e.g., Compound A, B, C, D, or E) of the present invention, or a pharmaceutically acceptable salt thereof.

In some embodiments, the present invention provides a method of treating B-cell prolymphocytic leukemia, comprising administering an MDM2 degrader (e.g., Compound A, B, C, D, or E) of the present invention, or a pharmaceutically acceptable salt thereof.

In some embodiments, the present invention provides a method of treating acute myeloid leukemia (AML), comprising administering an MDM2 degrader (e.g., Compound A, B, C, D, or E) of the present invention, or a pharmaceutically acceptable salt thereof.

In some embodiments, the present invention provides a method of treating Burkitt lymphoma/leukemia, comprising administering an MDM2 degrader (e.g., Compound A, B, C, D, or E) of the present invention, or a pharmaceutically acceptable salt thereof.

In some embodiments, the present invention provides a method of treating primary effusion lymphoma, comprising administering an MDM2 degrader (e.g., Compound A, B, C, D, or E) of the present invention, or a pharmaceutically acceptable salt thereof.

In some embodiments, the present invention provides a method of treating peripheral T-cell lymphoma (PTCL), comprising administering an MDM2 degrader (e.g., Compound A, B, C, D, or E) of the present invention, or a pharmaceutically acceptable salt thereof.

In some embodiments, the present invention provides a method of treating cutaneous T-cell lymphoma (CTCL), comprising administering an MDM2 degrader (e.g., Compound A, B, C, D, or E) of the present invention, or a pharmaceutically acceptable salt thereof.

In some embodiments, the present invention provides a method of treating diffuse large B-cell lymphoma (DLBCL), comprising administering an MDM2 degrader (e.g., Compound A, B, C, D, or E) of the present invention, or a pharmaceutically acceptable salt thereof.

In some embodiments, the present invention provides a method of treating advanced B-cell diffuse large B-cell lymphoma (ABC DLBCL), comprising administering an MDM2 degrader (e.g., Compound A, B, C, D, or E) of the present invention, or a pharmaceutically acceptable salt thereof.

In some embodiments, the present invention provides a method of treating intravascular large B-cell lymphoma, comprising administering an MDM2 degrader (e.g., Compound A, B, C, D, or E) of the present invention, or a pharmaceutically acceptable salt thereof.

In some embodiments, the present invention provides a method of treating lymphoplasmacytic lymphoma, comprising administering an MDM2 degrader (e.g., Compound A, B, C, D, or E) of the present invention, or a pharmaceutically acceptable salt thereof.

In some embodiments, the present invention provides a method of treating Waldenström's macroglobulinemia (WM), comprising administering an MDM2 degrader (e.g., Compound A, B, C, D, or E) of the present invention, or a pharmaceutically acceptable salt thereof.

In some embodiments, the present invention provides a method of treating splenic marginal zone lymphoma, comprising administering an MDM2 degrader (e.g., Compound A, B, C, D, or E) of the present invention, or a pharmaceutically acceptable salt thereof.

In some embodiments, the present invention provides a method of treating multiple myeloma, comprising administering an MDM2 degrader (e.g., Compound A, B, C, D, or E) of the present invention, or a pharmaceutically acceptable salt thereof.

In some embodiments, the present invention provides a method of treating plasmacytoma, comprising administering an MDM2 degrader (e.g., Compound A, B, C, D, or E) of the present invention, or a pharmaceutically acceptable salt thereof.

In some embodiments, the present invention provides a method of treating myelodysplastic syndrome (MDS), comprising administering an MDM2 degrader (e.g., Compound A, B, C, D, or E) of the present invention, or a pharmaceutically acceptable salt thereof.

In some embodiments, the present invention provides a method of treating malignant peripheral nerve sheath tumors (MPNST) in a patient in need thereof, comprising administering an MDM2 degrader (e.g., Compound A, B, C, D, or E) of the present invention, or a pharmaceutically acceptable salt thereof.

In some embodiments, the present invention provides a method of treating pancreatic cancer in a patient in need thereof, comprising administering an MDM2 degrader (e.g., Compound A, B, C, D, or E) of the present invention, or a pharmaceutically acceptable salt thereof.

In some embodiments, the present invention provides a method of treating primary CNS lymphomas in a patient in need thereof, comprising administering an MDM2 degrader (e.g., Compound A, B, C, D, or E) of the present invention, or a pharmaceutically acceptable salt thereof.

In some embodiments, the present invention provides a method of treating Hodgkin's lymphoma in a patient in need thereof, comprising administering an MDM2 degrader (e.g., Compound A, B, C, D, or E) of the present invention, or a pharmaceutically acceptable salt thereof.

In some embodiments, the present invention provides a method of treating primary cutaneous T-cell lymphoma in a patient in need thereof, comprising administering an MDM2 degrader (e.g., Compound A, B, C, D, or E) of the present invention, or a pharmaceutically acceptable salt thereof.

In some embodiments, the present invention provides a method of treating solid and liquid tumors in a patient in need thereof, comprising administering an MDM2 degrader (e.g., Compound A, B, C, D, or E) of the present invention, or a pharmaceutically acceptable salt thereof.

In some embodiments, the present invention provides a method of treating MYD88 mutant Waldenström macroglobulinemia in a patient in need thereof, comprising administering an MDM2 degrader (e.g., Compound A, B, C, D, or E) of the present invention, or a pharmaceutically acceptable salt thereof.

In some embodiments, the present invention provides a method of treating NSCLC in a patient in need thereof, comprising administering an MDM2 degrader (e.g., Compound A, B, C, D, or E) of the present invention, or a pharmaceutically acceptable salt thereof.

In some embodiments, the present invention provides a method of treating uveal melanoma in a patient in need thereof, comprising administering an MDM2 degrader (e.g., Compound A, B, C, D, or E) of the present invention, or a pharmaceutically acceptable salt thereof.

In some embodiments, the present invention provides a method of treating Ewing sarcoma in a patient in need thereof, comprising administering an MDM2 degrader (e.g., Compound A, B, C, D, or E) of the present invention, or a pharmaceutically acceptable salt thereof.

In some embodiments, the present invention provides a method of inhibiting the growth of a refractory (e.g., treatment resistant) tumor comprising administering an MDM2 degrader (e.g., Compound A, B, C, D, or E) of the present invention, or a pharmaceutically acceptable salt thereof. In some embodiments, the present invention provides a method of treating a refractory cancer comprising administering an MDM2 degrader (e.g., Compound A, B, C, D, or E) of the present invention, or a pharmaceutically acceptable salt thereof. In some embodiments, the refractory tumor include tumors that fail or are resistant to treatment with chemotherapeutic agents alone, radiation alone or combinations thereof. The cancer may become resistant at the beginning of treatment or it may become resistant during treatment. Refractory tumors lead to rapid disease progression, usually with poor prognosis. Examples of refractory tumors include carcinomas, gliomas, sarcomas, adenocarcinomas, adenosarcomas and adenomas. Such tumors occur in virtually all parts of the human body, including every organ. The tumors may, for example, be present in the breast, heart, lung, small intestine, colon, spleen, kidney, bladder, head and neck, ovary, prostate, brain, pancreas, skin, bone, bone marrow, blood, thymus, uterus, testicles, cervix, and liver.

In some embodiments, the present invention provides a method for the treatment of adult patients with a solid cancer or hematological malignancy who have received one prior therapy.

In some embodiments, the present invention provides a method for the treatment of adult patients with a solid cancer or hematological malignancy who have received two prior therapies.

In some embodiments, the present invention provides a method for the treatment of adult patients with a solid cancer or hematological malignancy who have received three prior therapies.

In some embodiments, the present invention provides a method for the treatment of adult patients with a solid cancer or hematological malignancy who have received at least one prior therapy.

In some embodiments, the present invention provides a method for the treatment of adult patients with a solid cancer or hematological malignancy who have received at least two prior therapies.

In some embodiments, the present invention provides a method for the treatment of adult patients with a solid cancer or hematological malignancy who have received at least three prior therapies.

In some embodiments, the present invention provides a method of increasing one or more protein markers (e.g., GDF15, p53, p21, and PUMA) in a patient in need thereof, comprising administering an MDM2 degrader (e.g., Compound A, B, C, D, or E) of the present invention, or a pharmaceutically acceptable salt thereof. In some embodiments, the method of increasing one or more protein markers (e.g., GDF15, p53, p21, and PUMA) comprises treating a solid cancer or hematological malignancy in a patient.

Combination Therapies

Depending upon the particular a solid cancer or hematological malignancy to be treated, additional therapeutic agents, which are normally administered to treat that condition, may be administered in combination with compounds and compositions of this invention. As used herein, additional therapeutic agents that are normally administered to treat a particular solid cancer or hematological malignancy, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: anti-inflammatory agents such as corticosteroids, TNF blockers, IL-1 RA, azathioprine, cyclophosphamide, and sulfasalazine; immunomodulatory and immunosuppressive agents such as cyclosporine, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketoconazole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided MDM2 degrader or a pharmaceutically acceptable salt thereof and one or more additional therapeutic agents. The therapeutic agent may be administered together with a provided MDM2 degrader or a pharmaceutically acceptable salt thereof, or may be administered prior to or following administration of a provided MDM2 degrader or a pharmaceutically acceptable salt thereof. Suitable therapeutic agents are described in further detail below. In certain embodiments, a provided MDM2 degrader or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided MDM2 degrader or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In some embodiments, a provided MDM2 degrader or a pharmaceutically acceptable salt thereof is administered to a patient at the doses and schedules provided herein and the one or more additional therapeutic agent is administered to the patient once every 1, 2, 3, 4, 5, 6, 7, 14, or 21 days.

In some embodiments, the additional therapeutical agent administered in combination with a provided MDM2 degrader or a pharmaceutically acceptable salt thereof is a BCL-2 inhibitor (e.g., venetoclax) or a MEK inhibitor (e.g., selumetinib) and is administered to the patient daily (QD). In some aspects, the BCL-2 inhibitor (e.g., venetoclax) or MEK inhibitor (e.g., selumetinib) is administered orally. In other aspects, the BCL-2 inhibitor (e.g., venetoclax) is administered a dose of about 5 mg/kg to about 20 mg/kg (e.g., about 7 mg/kg, about 8 mg/kg, about 9 mg/kg, or about 10 mg/kg). In other aspects, the MEK inhibitor (e.g., selumetinib) is administered a dose of about 0.01 mg/kg to about 5 mg/kg (e.g., about 0.1 mg/kg, about 0.5 mg/kg, or about 1 mg/kg).

In another embodiment, the present invention provides a method of treating a solid tumor comprising administering to a patient in need thereof a provided MDM2 degrader or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating diffuse large B-cell lymphoma (DLBCL) comprising administering to a patient in need thereof a provided MDM2 degrader or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In some embodiments, the present invention provides a method of treating a lymphoma comprising administering to a patient in need thereof a provided MDM2 degrader or a pharmaceutically acceptable salt thereof and a CHOP (cyclophosphamide, Hydrodaunorubicin®, Oncovin®, and prednisone or prednisolone) or R-CHOP (rituximab, cyclophosphamide, Hydrodaunorubicin®, Oncovin®, and prednisone or prednisolone) chemotherapy regimen.

In some embodiments, the present invention provides a method of treating a lymphoma comprising administering to a patient in need thereof a provided MDM2 degrader or a pharmaceutically acceptable salt thereof and a rituximab or bendamustine chemotherapy regimen.

In some embodiments, the present invention provides a method of treating a lymphoma comprising administering to a patient in need thereof a provided MDM2 degrader or a pharmaceutically acceptable salt thereof and a BTK inhibitor (e.g., ibrutinib).

In some embodiments, the present invention provides a method of treating a lymphoma comprising administering to a patient in need thereof a provided MDM2 degrader or a pharmaceutically acceptable salt thereof and an anti-CD20 antibody (e.g., rituximab).

In some embodiments, the present invention provides a method of treating a lymphoma comprising administering to a patient in need thereof a provided MDM2 degrader or a pharmaceutically acceptable salt thereof and an anti-CD79B ADC (e.g., polatuzumab).

In some embodiments, the present invention provides a method of treating a lymphoma comprising administering to a patient in need thereof a provided MDM2 degrader or a pharmaceutically acceptable salt thereof and a BCL-2 inhibitor (e.g., venetoclax).

In some embodiments, the present invention provides a method of treating leukemia (e.g., AML) comprising administering to a patient in need thereof a provided MDM2 degrader or a pharmaceutically acceptable salt thereof and a BCL-2 inhibitor (e.g., venetoclax). In some aspects of the method of treating leukemia (e.g., AML) with a combination of a provided MDM2 degrader and a BCL-2 inhibitor (e.g., venetoclax), the combination is additive. In some aspects of the method of treating leukemia (e.g., AML) with a combination of a provided MDM2 degrader and a BCL-2 inhibitor (e.g., venetoclax), the combination acts synergistically.

In some embodiments, the present invention provides a method of treating leukemia (e.g., AML) comprising administering to a patient in need thereof a provided MDM2 degrader or a pharmaceutically acceptable salt thereof and a BCL-2 inhibitor (e.g., venetoclax), wherein the lymphoma is resistant to treatment with the BCL-2 inihibitor (e.g., venetoclax) alone.

In some embodiments, the present invention provides a method of treating melanoma (e.g., uveal melanoma) comprising administering to a patient in need thereof a provided MDM2 degrader or a pharmaceutically acceptable salt thereof and a BCL-2 inhibitor (e.g., venetoclax). In some aspects of the method of treating melanoma (e.g., uveal melanoma) with a combination of a provided MDM2 degrader and a BCL-2 inhibitor (e.g., venetoclax), the combination is additive. In some aspects of the method of treating melanoma (e.g., uveal melanoma) with a combination of a provided MDM2 degrader and a BCL-2 inhibitor (e.g., venetoclax), the combination acts synergistically.

In some embodiments, the present invention provides a method of treating melanoma (e.g., uveal melanoma) comprising administering to a patient in need thereof a provided MDM2 degrader or a pharmaceutically acceptable salt thereof and a BCL-2 inhibitor (e.g., venetoclax), wherein the melanoma is resistant to treatment with the BCL-2 inihibitor (e.g., venetoclax) alone.

In some embodiments, the present invention provides a method of treating leukemia (e.g., AML) comprising administering to a patient in need thereof a provided MDM2 degrader or a pharmaceutically acceptable salt thereof and a FLT3 inhibitor (e.g., midostaurin). In some aspects of the method of treating leukemia (e.g., AML) with a combination of a provided MDM2 degrader and FLT3 inhibitor (e.g., midostaurin), the combination is additive. In some aspects of the method of treating leukemia (e.g., AML) with a combination of a provided MDM2 degrader and a FLT3 inhibitor (e.g., midostaurin), the combination acts synergistically.

In some embodiments, the present invention provides a method of treating leukemia (e.g., AML) comprising administering to a patient in need thereof a provided MDM2 degrader or a pharmaceutically acceptable salt thereof and a FLT3 inhibitor (e.g., midostaurin), wherein the lymphoma is resistant to treatment with the FLT3 inhibitor (e.g., midostaurin) alone.

In some embodiments, the present invention provides a method of treating a lymphoma comprising administering to a patient in need thereof a provided MDM2 degrader or a pharmaceutically acceptable salt thereof and lenalidomide or pomalidomide

In some embodiments, the present invention provides a method of treating a lymphoma comprising administering to a patient in need thereof a provided MDM2 degrader or a pharmaceutically acceptable salt thereof and a PI3K inhibitor (e.g., umbralisib).

In some embodiments, the present invention provides a method of treating a T-cell disease or deficiency describing herein comprising administering to a patient in need thereof a provided MDM2 degrader or a pharmaceutically acceptable salt thereof and a PI3K inhibitor (e.g., umbralisib).

In some embodiments, the present invention provides a method of treating a lymphoma comprising administering to a patient in need thereof a provided MDM2 degrader or a pharmaceutically acceptable salt thereof and a proteasome inhibitor (e.g., bortezomib).

In some embodiments, the present invention provides a method of treating a lymphoma comprising administering to a patient in need thereof a provided MDM2 degrader or a pharmaceutically acceptable salt thereof and chimeric antigen receptor T-cells.

In another embodiment, the present invention provides a method of treating multiple myeloma comprising administering to a patient in need thereof a provided MDM2 degrader or a pharmaceutically acceptable salt thereof and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström macroglobulinemia comprising administering to a patient in need thereof a provided MDM2 degrader or a pharmaceutically acceptable salt thereof and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, the present invention provides a method of treating a solid cancer or hematological malignancy in a patient in need thereof, comprising administering a therapeutically effective amount of the MDM2 degrader or a pharmaceutically acceptable salt thereof and an IDH inhibitor (e.g., AG120, AG221, BAY1436032, IDH305, etc.).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, the present invention provides a method of treating a solid cancer or hematological malignancy in a patient in need thereof, comprising administering a therapeutically effective amount of the MDM2 degrader or a pharmaceutically acceptable salt thereof and a apoptotic regulator (e.g., BCL-2 inhibitor).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFß trap compound (NCT02699515); and (NCT02517398). M7824 is comprised of a fully human IgG1 antibody against PD-L1 fused to the extracellular domain of human TGF-beta receptor II, which functions as a TGFß “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3 (e.g., sunitinib, lestaurtinib, tandutinib, crenolanib, gilteritinib, midostaurin, quizartinib, and sorafenib, FLX925, and G-749); Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, the present invention provides a method of treating a solid cancer comprising administering to a patient in need thereof a provided MDM2 degrader or a pharmaceutically acceptable salt thereof and azacitidine.

In some embodiments, the present invention provides a method of treating a solid cancer comprising administering to a patient in need thereof a provided MDM2 degrader or a pharmaceutically acceptable salt thereof and cytarabine.

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche), selumetinib (Koselugo™), binimetinib, trametinib, mirametinib (PD-325901), or TAK-733; trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

In some embodiments, the present invention provides a method of treating a solid cancer comprising administering to a patient in need thereof a provided MDM2 degrader or a pharmaceutically acceptable salt thereof and a MEK inhibitor (e.g., selumetinib).

In some embodiments, the present invention provides a method of treating a solid cancer or hematological malignancy in a patient in need thereof, comprising administering a therapeutically effective amount of the MDM2 degrader or a pharmaceutically acceptable salt thereof and a Flt3 inhibitor (e.g., midostaurin).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, 1Y5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating or lessening the severity of a disease comprising administering to a patient in need thereof a provided MDM2 degrader or a pharmaceutically acceptable salt thereof and a BTK inhibitor, wherein the disease is selected from B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenström macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis.

In another embodiment, the present invention provides a method of treating or lessening the severity of a disease comprising administering to a patient in need thereof a provided MDM2 degrader or a pharmaceutically acceptable salt thereof and a PI3K inhibitor, wherein the disease is selected from lymphomas, (including, for example, non-Hodgkin's Lymphoma (NHL) and Hodgkin's lymphoma (also termed Hodgkin's or Hodgkin's disease)).

In some embodiments, one or more other therapeutic agent is a phosphatidylinositol 3 kinase (PI3K) inhibitor selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3 (e.g., sunitinib, lestaurtinib, tandutinib, crenolanib, gilteritinib, midostaurin, quizartinib, and sorafenib, FLX925, and G-749); Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorn.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketoconazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof, see WO 2008/118802, US 2010/0197686), navitoclax (and analogs thereof, see U.S. Pat. No. 7,390,799), NH-1 (Shenayng Pharmaceutical University), obatoclax (and analogs thereof, see WO 2004/106328, US 2005/0014802), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C₁₋₁₀33, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-γ, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zamestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™), ); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan©), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided MDM2 degrader is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a solid cancer or hematological malignancy.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO 2011/070024, US 2011/0165156, WO 2011/0107553, US 2012/0329997, WO 2011/131407, US 2013/0005949, WO 2013/087699, US 2014/0336363, WO 2013/119716, WO 2013/132044, US 2014/0079706) or FPA-008 (WO 2011/140249, US 2011/0274683; WO 2013/169264; WO 2014/036357, US 2014/0079699).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO 2010/077634, US 2010/0203056), durvalumab (MED14736), BMS-936559 (WO 2007/005874, US 2009/0055944), and MSB0010718C (WO 2013/079174, US 2014/0341917).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO 2010/019570, US 2010/0150892, WO 2014/008218, US 2014/0093511), or IMP-731 or IMP-321 (WO 2008/132601, US 2010/0233183, WO 2009/044273, US 2011/0008331).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO 2006/105021, US 2007/0098719, WO 2009/009116, US 2009/0136494), or MK-4166 (WO 2011/028683, US 2012/0189639).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO 2009/073620, US 2011/053941, WO 2009/132238, US 2011/136796, WO 2011/056652, US 2012/277217, WO 2012/142237, US 2014/066625).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO 2006/029879, U.S. Pat. No. 7,501,496).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO 2011/109400, US 2013/0149236).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1 h68/GLV-1 h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682, the entirety of each of which is herein incorporated by reference, which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiment, an immuno-oncology agent is selected from the examples described in Table 1 of Jerry L. Adams ET. AL. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MED14736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MED10562 (Medimmune/AstraZeneca), an agonistic anti-OX40 antibody, in advanced solid tumors (NCT02318394 and NCT02705482); MED16469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations were performed under reduced pressure, preferably between about 15 mm Hg and 100 mm Hg (=20-133 mbar). The structure of final products, intermediates and starting materials was confirmed by standard analytical methods, e.g., microanalysis and spectroscopic characteristics, e.g., MS, IR, NMR. Abbreviations used are those conventional in the art.

All starting materials, building blocks, reagents, acids, bases, solvents, and catalysts utilized to synthesis the compounds of the present invention were either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions were carried out under nitrogen or argon unless otherwise stated.

Proton NMR (¹H NMR) was conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | BRUKER AVANCE III/400; Frequency (MHz) 400.13; Nucleus: 1H; |
| | Number of Transients: 8 |
| Prep-HPLC | Gilson GX-281 systems: instruments GX-A, GX-B, GX-C, GX-D, GX-E, |
| | GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase are used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃·H₂O in water (solvent A) and acetonitrile (solvent B).

HPLC Analytical Method: HPLC was carried out on X Bridge C18 150*4.6 mm, 5 micron. Column flow is 1.0 ml/min and mobile phase are used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5μ. Column flow was 16.0 ml/min. Mobile phase used was (A) 0.1% Formic Acid in Water and (B) Acetonitrile. Basic method used was (A) 5 mM ammonium bicarbonate and 0.10% NH₃ in Water and (B) Acetonitrile or (A) 0.10% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Intermediates

### [1-[(4-Methoxyphenyl) methyl]-2,6-dioxo-3-piperidyl] trifluoromethanesulfonate (Intermediate A)

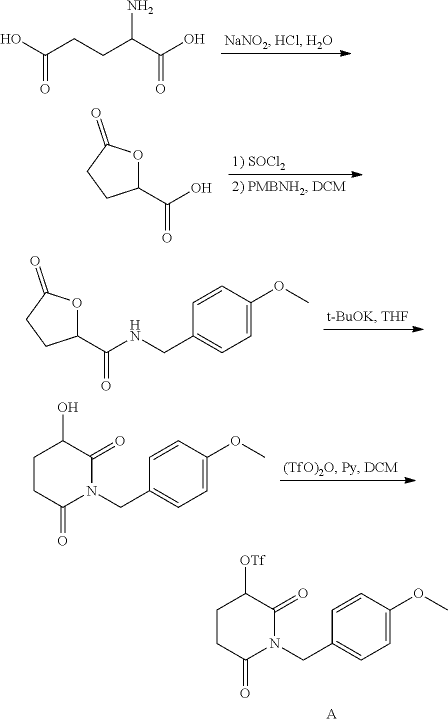

Step 1—5-Oxotetrahydrofuran-2-carboxylic acid. To a solution of 2-aminopentanedioic acid (210 g, 1.43 mol, CAS #617-65-2) in H₂O (800 mL) and HCl (12 M, 210 mL) was added a solution of NaNO₂ (147 g, 2.13 mol) in H₂O (400 mL) at −5° C. The mixture was stirred at 15° C. for 12 hrs. On completion, the mixture was concentrated and then dissolved in EA (500 mL) and filtered and washed with EA (3×100 mL). The filtrate and washed solution were dried over Na₂SO₄, filtered and concentrated in vacuo to give the title compound (200 g, crude) as yellow oil. ¹H NMR (400 MHz, CDCl₃) δ 6.43 (s, 1H), 5.02-4.95 (m, 1H), 2.67-2.38 (m, 4H)

Step 2—N-[(4-methoxyphenyl)methyl]-5-oxo-tetrahydrofuran-2-carboxamide. To 5-oxotetrahydrofuran-2-carboxylic acid (120 g, 922 mmol) was added SOCl₂ (246 g, 2.07 mol) at 0° C. slowly. The mixture was stirred at 85° C. for 3 hrs, and then the mixture was stirred at 15° C. for 6 hrs. The mixture was concentrated in vacuo. The residue was dissolved in dry DCM (1 L) at 0° C. under N₂. After that a solution of Et₃N (187 g, 1.84 mol) and 4-methoxybenzylamine (101 g, 738 mmol) in DCM (400 mL) was added, then the mixture was stirred at 15° C. for 3 hrs. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. ¹H NMR (400 MHz, CDCl₃) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI⁺) m/z 272.0 (M+Na)⁺.

Step 3—3-Hydroxy-1-[(4-methoxyphenyl)methyl]piperidine-2,6-dione. A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C. under nitrogen atmosphere. The resulting reaction mixture was stirred at −40° C. for 1 hr. On completion, the reaction mixture was quenched with saturated NH₄Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. ¹H NMR (400 MHz, CDCl₃) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

Step 4—[1-[(4-Methoxyphenyl) methyl]-2,6-dioxo-3-piperidyl] trifluoromethanesulfonate. To a solution of 3-hydroxy-1-[(4-methoxyphenyl) methyl] piperidine-2, 6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C. for 1.5 hours under N₂. On completion, the mixture was concentrated in vacuo. The residue was purified by column chromatography on silica gel (PE:EA=20:1/8:1) to give the title compound (45.0 g, 68% yield) as light yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### 5-Bromo-3-methyl-1H-benzimidazol-2-one (Intermediate D)

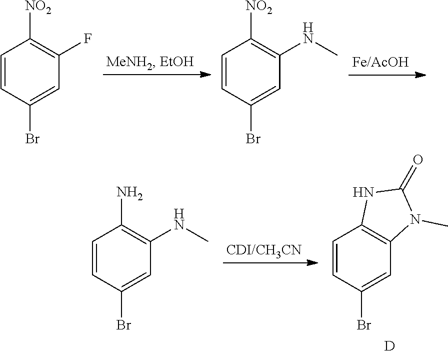

Step 1—5-Bromo-N-methyl-2-nitro-aniline. 4-bromo-2-fluoro-1-nitro-benzene (230 g, 1.05 mol, CAS #321-23-3) was added to a solution of mehylamine in tetrahydrofuran (2 M, 1.51 L). The mixture was stirred at 15° C. for 10 minutes. On completion, the mixture was diluted with H₂O (250 mL) and extracted with EtOAc (3×300 mL). The combined organic layers were washed with brine (300 mL), dried over Na₂SO₄, filtered and concentrated in vacuo to give the title compound (200 g, 83% yield) as a yellow solid. ¹H NMR (400 MHz, DMSO-d₆) δ 8.22 (s, 1H), 7.98 (d, J=9.2 Hz, 1H), 7.16 (d, J=1.6 Hz, 1H), 6.82 (dd, J=8.4, 1.6 Hz, 1H), 2.95 (d, J=4.8 Hz, 3H).

Step 2—4-Bromo-N2-methyl-benzene-1,2-diamine. To a mixture of 5-bromo-N-methyl-2-nitro-aniline (200 g, 865 mmol) in EtOAc (1 L) and H₂O (500 mL) was added AcOH (1.00 L). The mixture was warmed to 50° C., and then Fe (174 g, 3.11 mol) was added to the reaction mixture. After that, the reaction mixture was stirred at 80° C. for 6 hours. On completion, the mixture was filtered through celite. The filtrate was concentrated in vacuo and the residue was diluted with H₂O (250 mL) and extracted with EtOAc (3×300 mL). The combined organic layers were washed with aq.NaHCO₃ and brine (300 mL), dried over Na₂SO₄, filtered and concentrated in vacuo. The residue was purified by flash silica gel chromatography to give the title compound (130 g, 75% yield) as black oil. ¹H NMR (400 MHz, DMSO-d₆) δ 6.55-6.52 (m, 1H), 6.48-6.45 (m, 1H), 6.43-6.42 (m, 1H), 4.89-4.88 (m, 1H), 4.61 (s, 2H), 2.70 (d, J=4.0 Hz, 3H).

Step 3—5-Bromo-3-methyl-1H-benzimidazol-2-one. To a solution of 4-bromo-N2-methyl-benzene-1,2-diamine (110 g, 547 mmol) in CH₃CN (1.3 L) was added CDI (177 g, 1.09 mol). The mixture was stirred at 80° C. for 6 hours under N₂. On completion, the mixture was concentrated in vacuo. The mixture was diluted with H₂O (1.0 L) and filtered. The filter cake was washed with water (3×200 mL) and dried in vacuo to give the title compound (106 g, 85% yield) as a white solid. ¹H NMR (400 MHz, DMSO-d₆) δ 11.00 (s, 1H), 7.33 (s, 1H), 7.13 (d, J=8.0 Hz, 1H), 6.92 (d, J=8.0 Hz, 1H), 3.27 (s, 3H).

### 3-(5-bromo-3-methyl-2-oxo-benzimidazol-1-yl)piperidine-2,6-dione (3-(5-bromo-3-methyl-2-oxo-2,3-dihydro-1H-1,3-benzodiazol-1-yl)piperidine-2,6-dione) (Intermediate E)

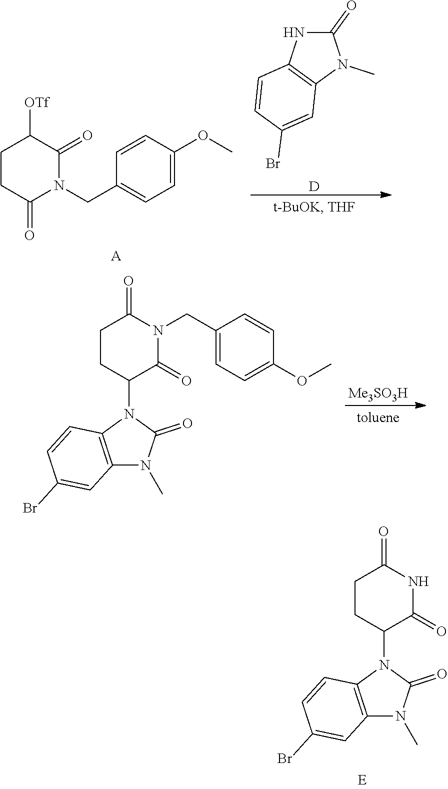

Step 1—3-(5-Bromo-3-methyl-2-oxo-benzimidazol-1-yl)-1-[(4-methoxyphenyl)methyl]piperidine-2,6-dione. To a solution of 5-bromo-3-methyl-1H-benzimidazol-2-one (4.90 g, 21.6 mmol, Intermediate D) in THF (300 mL) was added t-BuOK (3.63 g, 32.3 mmol) at 0° C. The mixture was stirred at 0-10° C. for 1 hour under N₂. Then a solution of [1-[(4-methoxyphenyl) methyl]-2, 6-dioxo-3-piperidyl] trifluoromethanesulfonate (9.87 g, 25.9 mmol, Intermediate A) in THF (100 mL) was added to the reaction mixture at 0-10° C. during 30 minutes. The mixture was stirred at 0-10° C. for 30 minutes under N₂. An additional solution of [1-[(4-methoxyphenyl)methyl]-2,6-dioxo-3-piperidyl]trifluoromethanesulfonate (2.47 g, 6.47 mmol) in THF (20 mL) was added to the reaction mixture at 0-10° C. dropwise. The mixture was then stirred at 0-10° C. for another 30 minutes under N₂. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch title compound (1.80 g, 18% yield) as light yellow solid. ¹H NMR (400 MHz, DMSO-d₆) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

Step 2—3-(5-Bromo-3-methyl-2-oxo-benzimidazol-1-yl)piperidine-2,6-dione. To a mixture of 3-(5-bromo-3-methyl-2-oxo-benzimidazol-1-yl)-1-[(4-methoxyphenyl)methyl] piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C. for 2 hours. On completion, the reaction mixture was cooled to room temperature and concentrated in vacuo. The residue was poured into ice/water (200 mL), and extracted with EA (3×100 mL). The combined organic layer was washed with brine (50 mL), dried over Na₂SO₄, filtered and concentrated in vacuo. The residue was triturated with EA (80 mL) and filtered. The filtrate cake was collected and dried in vacuo to give the title compound (4.20 g, 67% yield) as off-white solid. ¹H NMR (400 MHz, DMSO-d₆) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m, 1H), 2.34 (s, 3H), 2.92-2.88 (m, 1H), 2.71-2.60 (m, 2H), 2.03-1.99 (m, 1H).

### 3-[3-Methyl-2-oxo-5-(4-piperidyl)benzimidazol-1-yl]piperidine-2,6-dione (Intermediate F)

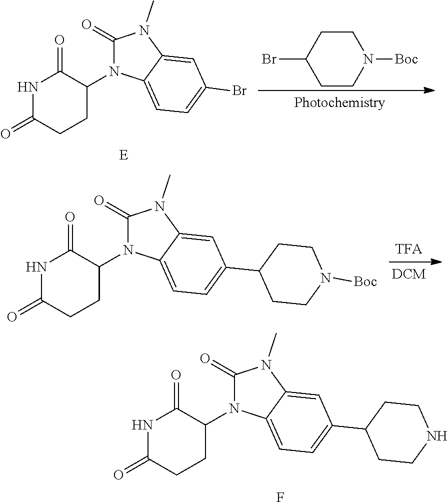

Step 1—Tert-butyl 4-[1-(2,6-dioxo-3-piperidyl)-3-methyl-2-oxo-benzimidazol-5-yl]piperidine-1-carboxylate. To an 40 mL vial equipped with a stir bar was added 3-(5-bromo-3-methyl-2-oxo-benzimidazol-1-yl)piperidine-2,6-dione (1.00 g, 2.96 mmol, Intermediate E), tert-butyl 4-bromopiperidine-1-carboxylate (1.02 g, 3.84 mmol, CAS #180695-79-8), Ir[dF(CF₃)ppy]₂(dtbpy)(PF₆) (33.18 mg, 29.57 umol), NiCl₂.dtbbpy (5.88 mg, 14.7 umol), TTMSS (735 mg, 2.96 mmol), 2,6-dimethylpyridine (633.75 mg, 5.91 mmol) in DME (15 mL). The vial was sealed and placed under nitrogen. The reaction was stirred and irradiated with a 50W [455 nm] blue LED lamp (3 cm away), with cooling water to keep the reaction temperature at 25° C. for 14 hours. On completion, the mixture was filtered and concentrated to give a residue. The residue was purified by column chromatography (SiO₂, DCM/Ethyl acetate=1:0 to 1:1) to give the title compound (1.13 g, 86% yield) as a yellow solid. ¹H NMR (400 MHz, DMSO-d6) δ 11.18-10.94 (m, 1H), 7.11 (s, 1H), 7.01 (d, J=8.0 Hz, 1H), 6.91 (dd, J=0.8, 8.0 Hz, 1H), 5.75 (s, 1H), 5.33 (dd, J=5.6, 12.8 Hz, 1H), 4.09 (d, J=11.2 Hz, 2H), 3.33 (s, 3H), 2.95-2.83 (m, 2H), 2.76-2.57 (m, 4H), 1.75 (d, J=12.0 Hz, 2H), 1.55 (dq, J=4.0, 12.4 Hz, 2H), 1.42 (s, 9H); LC-MS (ESI+) m/z 443.2 (M+H)⁺.

Step 2—3-[3-Methyl-2-oxo-5-(4-piperidyl)benzimidazol-1-yl]piperidine-2,6-dione. To a solution of tert-butyl 4-[1-(2,6-dioxo-3-piperidyl)-3-methyl-2-oxo-benzimidazol-5-yl]piperidine-1-carboxylate (150 mg, 338 umol) in DCM (3 mL) was added TFA (773 mg, 6.78 mmol). The mixture was stirred at 25° C. for 0.5 hour. On completion, the mixture was concentrated to give the title compound (150 mg, 96.95% yield, TFA salt) as a colorless oil; LC-MS (ESI+) m/z 343.1 (M+H)⁺.

### Chloro-(3-chloro-2-fluoro-phenyl)-oxo-dispiro[BLAH]carboxylic acid (Intermediate G)

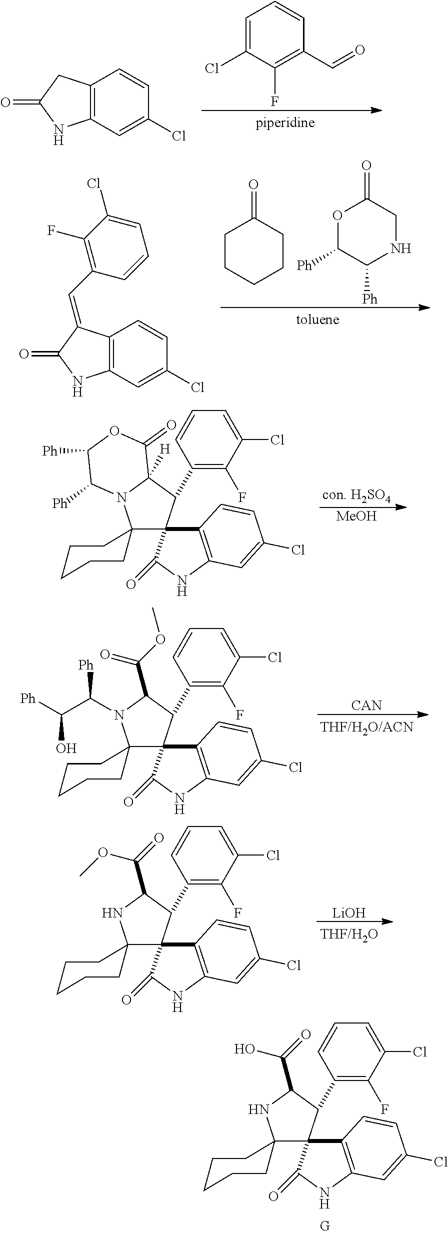

Step 1—(3E)-6-chloro-3-[(3-chloro-2-fluoro-phenyl)methylene]indolin-2-one. A 500 mL 3-necked round bottom flask was charged with 6-chloroindolin-2-one (89.6 g, 535 mmol, CAS #56341-37-8), 3-chloro-2-fluoro-benzaldehyde (84.8 g, 535 mmol, CAS #85070-48-0), MeOH (1700 mL) and piperidine (9.11 g, 107 mmol). The mixture was stirred at 65° C. for 5 h, then at 25° C. for 12 h. On completion, the reaction mixture was filtered and the filter cake was dried under reduced pressure to give title product (160 g, 94% yield). ¹H NMR (400 MHz, DMSO-d₆) δ=10.87 (s, 1H), 7.82-7.63 (m, 2H), 7.56 (s, 1H), 7.39 (t, J=8.0 Hz, 1H), 7.18 (d, J=8.0 Hz, 1H), 7.03-6.77 (m, 2H).

Step 2—chloro-(3-chloro-2-fluoro-phenyl)-diphenyl-dispiro[BLAH]dione. (3E)-6-chloro-3-[(3-chloro-2-fluoro-phenyl)methylene]indolin-2-one (50 g, 162 mmol), (5R,6S)-5,6-diphenylmorpholin-2-one (49.3 g, 194 mmol, CAS #282735-66-4), and cyclohexanone (31.8 g, 324 mmol, 33.6 mL) were dissolved in THF (75 mL) and toluene (750 mL) and 140° C. for 12 hours. On completion, the reaction mixture was concentrated in vacuo to give a residue. The residue was purified by column chromatography (SiO₂, petroleum ether/ethyl acetate=8/1 to 5/1) to give the title compound (160 g 97% purity). ¹H NMR (400 MHz, DMSO-d₆) δ=10.79 (s, 1H), 7.95 (t, J=6.8 Hz, 1H), 7.45-7.37 (m, 1H), 7.33-7.20 (m, 4H), 7.18-7.09 (m, 4H), 7.07-6.98 (m, 2H), 6.86-6.75 (m, 3H), 6.66 (dd, J=2.0, 8.4 Hz, 1H), 6.35 (d, J=8.4 Hz, 1H), 5.44 (d, J=11.2 Hz, 1H), 4.90 (d, J=2.8 Hz, 1H), 4.58 (d, J=11.2 Hz, 1H), 2.39 (d, J=12.8 Hz, 1H), 2.24-2.09 (m, 1H), 1.42-1.18 (m, 4H), 1.10-0.78 (m, 1H).

Step 3—methyl chloro-(3-chloro-2-fluoro-phenyl)-[(1R,2S)-2-hydroxy-1,2-diphenyl-ethyl]-oxo-dispiro[BLAH]carboxylate. H₂SO₄ (9.07 g, 92.5 mmol, 4.93 mL) was added to a solution of intermediate chloro-(3-chloro-2-fluoro-phenyl)-diphenyl-dispiro[BLAH]dione (9.0 g, 14.03 mmol) dissolved in MeOH (70 mL) and the resulting solution was heated to 50° C. for 5 hours. On completion, the reaction mixture was cooled to 0° C. and slowly neutralized with a solution of saturated sodium bicarbonate. The aqueous solution was extracted with ethyl acetate, and the organic layer was dried over sodium sulfate, filtered, concentrated to give the residue. The residue was purified by reverse phase flash [ACN/(0.1% FA in water), 0% to 90%] to give title compound (7.0 g 84.2% purity). ¹H NMR (400 MHz, DMSO-d₆) δ=7.74-7.68 (m, 1H), 7.57 (s, 1H), 7.51 (d, J=8.4 Hz, 1H), 7.41 (d, J=7.2 Hz, 4H), 7.25 (d, J=7.6 Hz, 6H), 7.19-7.11 (m, 6H), 7.10-6.98 (m, 4H), 6.94-6.88 (m, 1H), 6.65-6.58 (m, 1H), 5.39-5.27 (m, 1H), 4.89-4.75 (m, 1H), 4.42-4.29 (m, 2H), 4.04 (q, J=6.8 Hz, 1H), 3.63-3.53 (m, 2H), 3.40 (s, 3H), 2.22-2.12 (m, 1H), 2.05-1.94 (m, 3H), 1.40-1.32 (m, 2H), 1.28-1.13 (m, 3H).

Step 4—methyl chloro-(3-chloro-2-fluoro-phenyl)-oxo-dispiro[BLAH]carboxylate. The resulting intermediate methyl chloro-(3-chloro-2-fluoro-phenyl)-[(1R,2S)-2-hydroxy-1,2-diphenyl-ethyl]-oxo-dispiro[BLAH]carboxylate (7.0 g, 10.3 mmol) was dissolved in ACN (78 mL), then CAN (11.3 g, 20.7 mmol) was added, followed by the addition of H₂O (78 mL). The reaction was stirred at 25° C. for 30 min. On completion, the reaction mixture was quenched by adding the mixture to a cold saturated aqueous NaHCO₃ solution (50 mL). The aqueous layer was extracted with ethyl acetate (20 mL×3). The organic layer was separated, dried over anhydrous Na₂SO₄, filtered and concentrated under reduced pressure to give the residue. The residue was purified by column chromatography (SiO₂, petroleum ether/ethyl acetate=50/1 to 5/1) to give title compound (1.58 g, 31% purity). LC-MS (ESI⁺) m/z 477.2 (M+H)⁺.

Step 5—chloro-(3-chloro-2-fluoro-phenyl)-oxo-dispiro[BLAH]carboxylic acid. Methyl chloro-(3-chloro-2-fluoro-phenyl)-oxo-dispiro[BLAH]carboxylate (2.00 g, 4.19 mmol) was dissolved in THF (14 mL) and LiOH·H₂O (527 mg, 12.5 mmol) was added followed by water (14 mL) and MeOH (2 mL) and the reaction was stirred at 25° C. for 15 min. On completion, water (20 mL) was added and the reaction was slowly neutralized with 2M HCl and the suspension was stirred for 15 min. The resulting precipitate was filtered, washed with water to give title compound (1.50 g, 70% yield). ¹H NMR (400 MHz, DMSO-d₆) δ=10.75-10.57 (m, 1H), 10.55 (s, 1H), 7.61-7.54 (m, 1H), 7.50-7.44 (m, 1H), 7.41-7.34 (m, 1H), 7.18-7.12 (m, 1H), 7.08-7.02 (m, 1H), 6.72-6.66 (m, 1H), 4.72-4.65 (m, 1H), 4.54-4.47 (m, 1H), 3.18-3.15 (m, 1H), 2.22-2.13 (m, 1H), 1.83-1.70 (m, 2H), 1.64-1.52 (m, 3H), 1.51-1.43 (m, 2H), 1.42-1.34 (m, 1H), 1.04-0.92 (m, 1H), 0.89-0.77 (m, 1H). LC-MS (ESI⁺) m/z 463.2 (M+H)⁺.

### tert-Butyl N-[4-[4-[1-(2,6-dioxo-3-piperidyl)-3-methyl-2-oxo-benzimidazol-5-yl]piperidine-1-carbonyl]cyclohexyl]carbamate (Intermediate B)

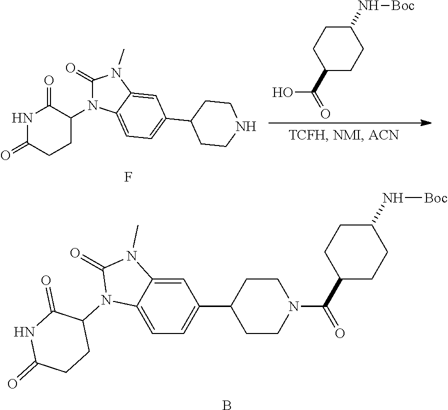

To a solution of 3-[3-methyl-2-oxo-5-(4-piperidyl)benzimidazol-1-yl]piperidine-2,6-dione (4.30 g, 12.5 mmol) and 4-(tert-butoxycarbonylamino)cyclohexanecarboxylic acid (3.67 g, 15.0 mmol, CAS #130309-46-5) in ACN (80 mL) was added 1-methylimidazole (33.0 g, 401 mmol) and [chloro(dimethylamino)methylene]-dimethyl-ammonium; hexafluorophosphate (10.5 g, 37.6 mmol) at 25° C. The reaction solution was stirred at 25° C. for 2 minutes (the reaction was complete once the reaction system became clear). On completion, the reaction mixture was concentrated in vacuo. The concentrated residue was purified by reverse phase flash [CAN/(0.1% TFA in water), 0% to 90%] to give the title compound (6.70 g, 84% yield) as a white solid. LC-MS (ESI+) m/z 568.4 (M+H)⁺.

### 3-[5-[1-(4-Aminocyclohexanecarbonyl)-4-piperidyl]-3-methyl-2-oxo-benzimidazol-1-yl]piperidine-2,6-dione (Intermediate C)

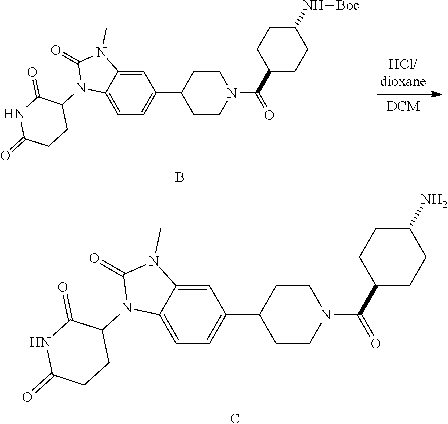

To a mixture of tert-butyl N-[4-[4-[1-(2,6-dioxo-3-piperidyl)-3-methyl-2-oxo-benzimidazol-5-yl] piperidine-1-carbonyl]cyclohexyl]carbamate (5.70 g, 10.0 mmol) in DCM (5 mL) was added HCl/dioxane (4 M, 58 mL) in one portion at 25° C. under N₂. The mixture was stirred at 25° C. for 30 minutes. On completion. The reaction mixture was concentrated in vacuo to give the title compound (4.69 g, crude, HCl salt) as a white solid. LC-MS (ESI+) m/z 468.3 (M+H)⁺.

### Example 1. Synthesis of (3′R,4'S,5′R)-6″-chloro-4′-(3-chloro-2-fluorophenyl)-N-((1r,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide (“Compound A”)

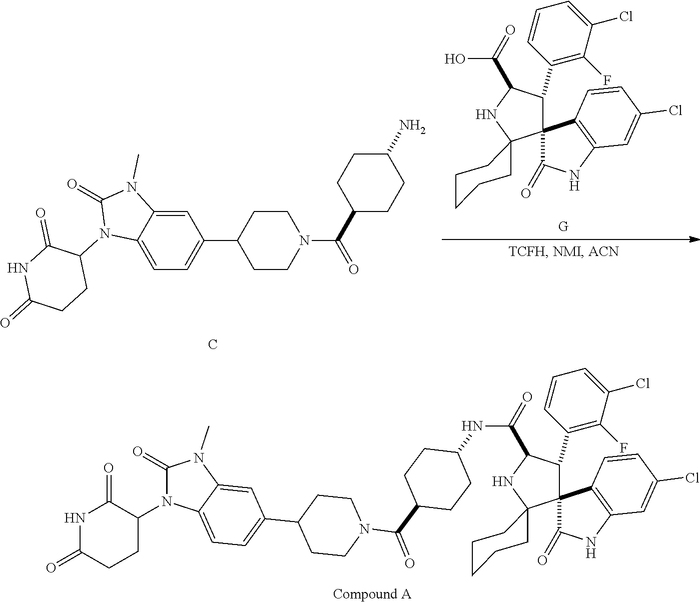

To a solution of 3-[5-[1-(4-aminocyclohexanecarbonyl)-4-piperidyl]-3-methyl-2-oxo-benzimidazol-1-yl]piperidine-2,6-dione (Intermediate C, 4.24 g, 9.06 mmol) and chloro-(3-chloro-2-fluoro-phenyl)-oxo-dispiro[BLAH] carboxylic acid (Intermediate G, 3.50 g, 7.55 mmol) in ACN (100 mL) was added [chloro(dimethylamino)methylene]-dimethyl-ammonium; hexafluorophosphate (6.36 g, 22.6 mmol) and 1-methylimidazole (19.8 g, 241 mmol) at 25° C. The reaction solution was stirred at 25° C. for 2 minutes (the reaction was complete once the reaction system became clear). On completion, the reaction mixture was concentrated in vacuo. The concentrated residue was purified by reverse phase flash [ACN/(0.1% FA in water), 0% to 90%] to give the title compound (5.10 g, 72% yield) as a white solid. ¹H NMR (400 MHz, DMSO-d₆) δ 11.08 (s, 1H), 10.52 (s, 1H), 7.77 (d, J=8.0 Hz, 1H), 7.58 (t, J=6.8 Hz, 1H), 7.41 (d, J=8.0 Hz, 1H), 7.32 (t, J=7.2 Hz, 1H), 7.15-7.08 (m, 2H), 7.06-6.98 (m, 2H), 6.92 (d, J=8.0 Hz, 1H), 6.67 (s, 1H), 5.34 (dd, J=5.2, 12.7 Hz, 1H), 4.56 (d, J=9.2 Hz, 2H), 4.37 (d, J=9.2 Hz, 1H), 4.09 (br d, J=11.6 Hz, 1H), 3.55-3.45 (m, 1H), 3.36-3.33 (m, 3H), 3.16-3.05 (m, 1H), 2.94-2.84 (m, 1H), 2.80 (t, J=12.0 Hz, 1H), 2.75-2.67 (m, 1H), 2.64 (s, 1H), 2.60 (s, 1H), 2.56-2.52 (m, 1H), 2.05-1.91 (m, 2H), 1.90-1.82 (m, 2H), 1.81-1.70 (m, 5H), 1.59 (s, 4H), 1.48 (d, J=13.6 Hz, 4H), 1.41-1.28 (m, 4H), 1.00-0.74 (m, 2H). LC-MS (ESI+) m/z 912.4 (M+H)⁺

Compounds B, C, D, and E can be prepared by similar methods as Compound A or as disclosed in WO 2021/188948, the contents of which are incorporated herein by reference in their entireties.

### Example 2. In Vitro and In Vivo Experiments

Compound A was tested in MDM2-dependent cell lines in vitro, as well as in sub-cutaneous and disseminated mouse AML and ALL xenograft models. Methods included in vitro cell proliferation and apoptosis assays, gene expression profiling, and in vivo pharmacological studies.

Cell Lines:

All cell lines (RS4;11, MOLM-13, MV-4-11, OCI-Ly10, OCI-Ly19, Kasumi-1, 92-1, Mel202, SK-MEL-28) were procured from ATCC (https://www.atcc.org), ECACC (https://www.sigmaaldrich.com) or DSMZ (https://www.dsmz.de) and cultured in recommended culture medium at 37° C., 5% CO₂. Identity of each cell line was confirmed by 48-variant SNP panel and were confirmed mycoplasma free.

In Vitro Cell Viability and Apoptosis:

In vitro cell viability in all cells lines, at optimized growth conditions, was assessed using Cell Titer Glo (Promega) assay performed over 24 h-96 h compound treatments. Compound treatments were carried out using a 11-point dose response, 3-fold serial dilutions stating at a maximum concentration of 1 uM. IC₅₀, IC₉₀ values were calculated by curve fitting using appropriate Nonlinear Regression fit in GraphPad Prism. Cell cycle arrest and/or apoptosis in response to single agent or combination treatment was assessed using flow cytometry analysis on cells treated for 24 h.

For washout assay, cells were treated for 4-24 h at serial dilutions indicated above. At the end of treatment times (4 h, 8 h, 16 h and 24 h) cells were washed twice with complete media, plated in 384-well plates and incubated for 24-72 h. Cells were assessed for viability using Cell Titer Glo (Promega) assay and apoptosis using Caspase Glo 3/7 (Promega) assay at 24 h, 48 h and 72 h post the start of treatment.

In Vivo Studies with Subcutaneous Xenografts:

Female mice, 5-8 wks old mice NOD/SCID mice from SPF (Beijing) experimental Animal Science Technology Co., ltd. Animals were received, housed and observed for seven days prior to study initiation. Each mouse were inoculated subcutaneously with RS4;11 tumor cells (1×107) in 0.1 ml of RPMI1640 with 10% FBS for tumor development. Groups and treatments were started when the mean tumor volume reached about 100 mm3 for study. Based on the tumor volume, mice were randomly assigned to respective groups using a computer-generated randomization procedure. Compound A was formulated in 20% HP-β-CD at concentrations up to 0.1 mg/mL and administered intravenously to tumor bearing animals. Tumor size was measured twice to three times weekly in three dimensions using a caliper. The measurement of tumor size were conducted twice weekly with a caliper and recorded. The tumor volume (mm³) was estimated using the formula: TV=a×b2/2, where “a” and “b” are long and short diameters of a tumor, respectively.

At various times tumor bearing animals were sacrificed and plasma and tumors collected for pharmacokinetic and pharmacodynamic determinations.

In Vivo Studies with Disseminated Xenografts:

Female mice, 5-8 wks old mice NOD/SCID mice from SPF (Beijing) experimental Animal Science Technology Co., ltd. Animals were received, housed and observed for seven days prior to study initiation. Each mouse will be with tumor cells in 0.1 ml PBS via tail-vein injection. Groups and treatments were started 4 days post-inoculation. Based on the body weight, mice were randomly assigned to respective groups using a computer-generated randomization procedure. Compound A was formulated in 20% HP-β-CD at concentrations up to 0.1 mg/mL and administered intravenously to tumor bearing animals.

PD Assessment in In Vitro and In Vivo Samples:

TaqMan™ Array, Human p53-Mediated Apoptosis (Thermo Fisher Scientific, 4418812) and, TaqMan™ Array, Human p53 Signaling (Thermo Fisher Scientific, 4418813) were used to identify downstream markers of change in p53 activity. A short list of 8 markers (MDM2, GDF15, CDKN1A, GADD45A, TNFRSF10B, FAS, BBC3, BAX) were validated and used for PD assessment. Catalogued Taqman assays for all markers were obtained from Thermo Fisher Scientific.

In Vivo AML Experiments

AML patient cells from leukapheresis were intravenously (IV) injected and established in immunocompromised host strain mice. Surrogate animals were used to determine the level of engraftment targeting a threshold of ˜20% huCD45+ cells in BM. Mice were randomized on study and treatment was initiated. Vehicle and Compound A, 1 mg/kg were administered IV, every three weeks for six weeks (total of two doses of Compound A). At study end, whole blood, bone marrow and spleen were assessed by flow cytometry.

Results:

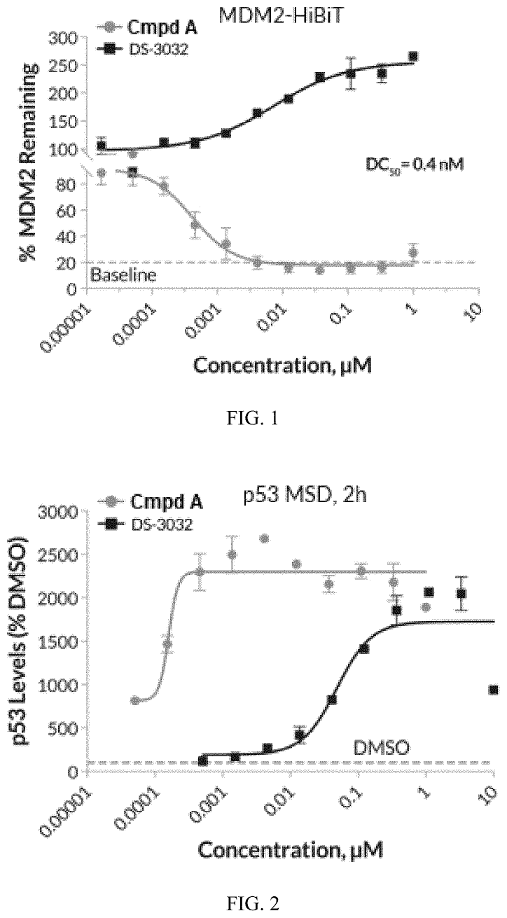

FIG. 1 shows that Compound A shows rapid and potent degradation of MDM2. 293T cells stably expressing C-terminally HiBiT-tagged MDM2 were treated for 4 h with Compound A or small molecule inhibitor DS-3032 in a 3-fold serial dilution dose-response at maximum concentration of 1 mM. Degradation potency was measured as reduction in HiBiT signal, quantified using Nano-Glo® HiBiT lytic detection system (Promega).

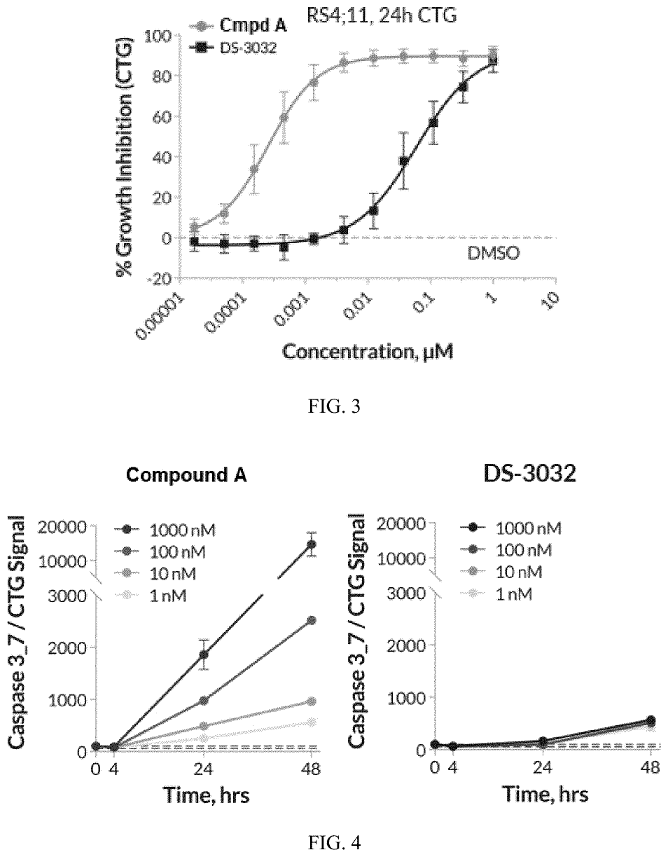

FIG. 2 shows that Compound A of cancer cells shows strong stabilization of p53. Acute lymphoblastic leukemia cells (RS4;11) were treated for 2 h (4 h, 8 h and 24 h—data not shown) with Compound A or small molecule inhibitor DS-3032 in a 3-fold serial dilution dose-response at maximum concentration of 1 mM. p53 levels were quantified using p53 MSD assay (Total p53 Whole Cell Lysate Kit, MSD, K150DBD-2).

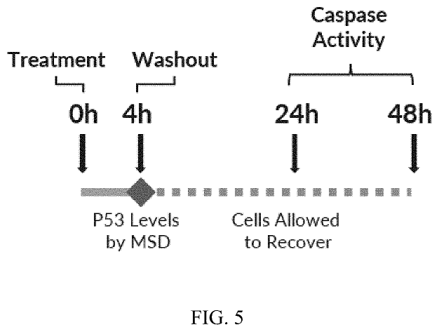

FIG. 3 shows that Compound A, compared with SMI, potently inhibits cancer cell growth. Acute lymphoblastic leukemia cells (RS4;11) were treated for 24 h with Compound A or small molecule inhibitor DS-3032 in a 3-fold serial dilution dose-response at maximum concentration of 1 mM. Growth inhibition was quantified using Cell titer glo (Promega) assay at 24 h continuous treatment.

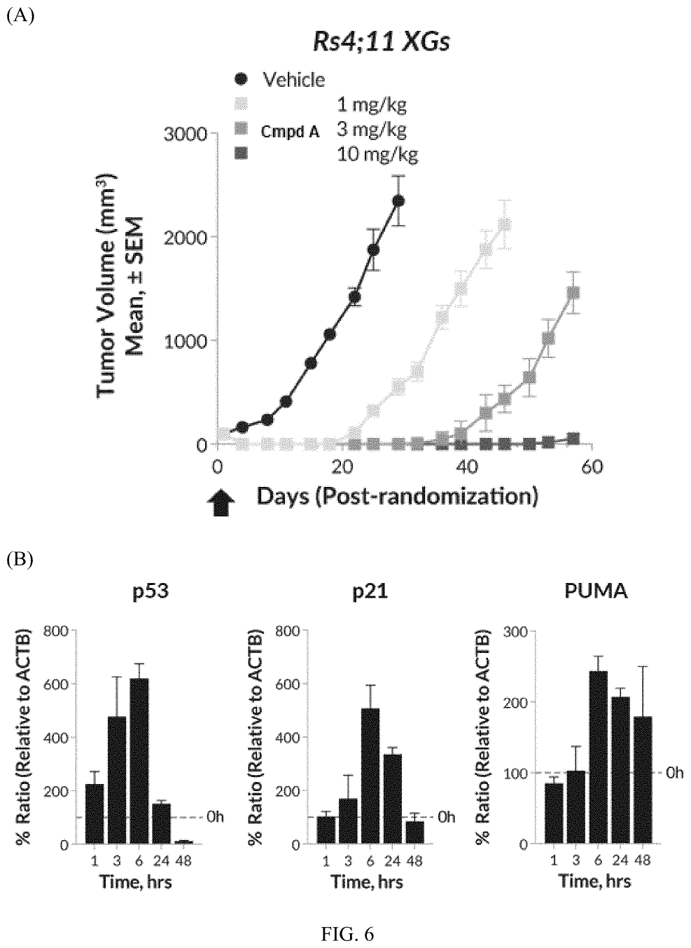

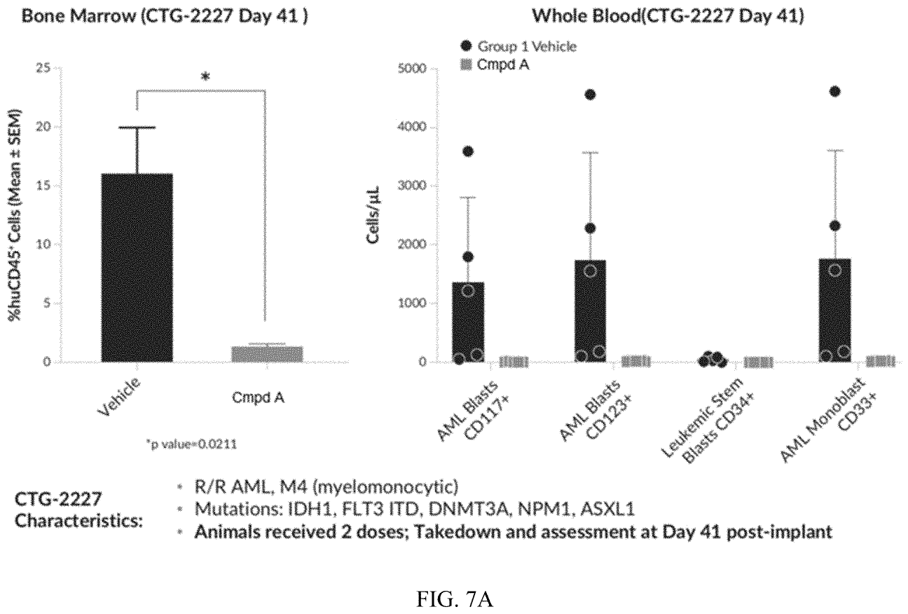

FIG. 4 shows that short term exposure to Compound A is sufficient to induce apoptosis. Acute lymphoblastic leukemia cells (RS4;11) were treated for 4 h (8 h, 16 h, 24 h—data not shown) with Compound A or small molecule inhibitor DS-3032 at indicated concentrations. Apoptotic activity was quantified as increase in Caspase activity measured using Caspase Glo® 3/7 assay system (Promega). Compared with DS-3032, a short 4 h exposure to Compound A followed by washout was sufficient to potently induce apoptosis in cancer cells measured at 24 or 48 h post treatment. FIG. 5 shows a schematic of the washout experiments performed in RS4;11 cells.

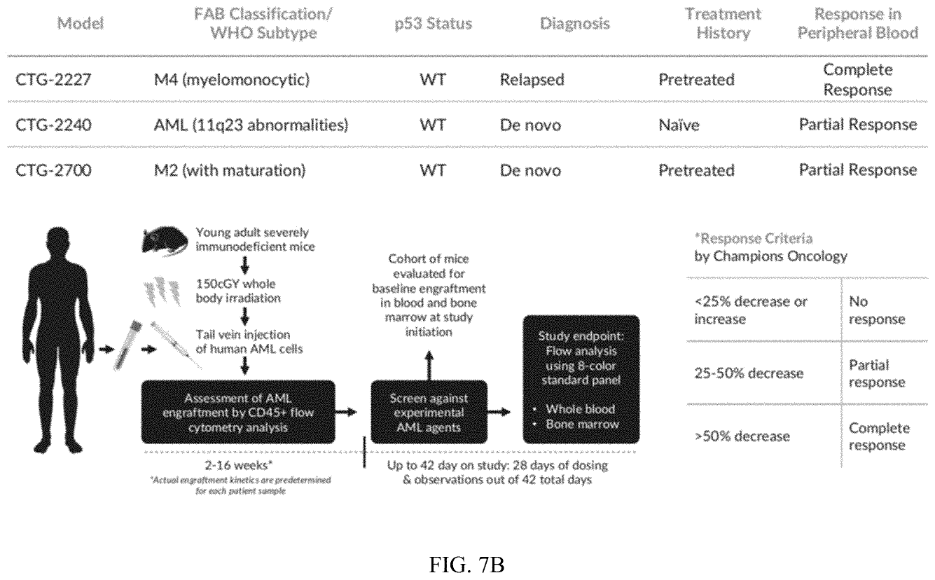

Compound A was administered as a single intravenous bolus in RS411 tumor bearing mice at doses of 1, 3, and 10 mg/kg. In panel A, FIG. 6 shows that Compound A demonstrated a dose-responsive, anti-tumor activity that led to sustained tumor regression at doses as low as 1 mg/kg. In panel B, FIG. 6 shows that MDM2 degradation with Compound A at 1 mg/kg led to marked upregulation of protein markers, such as but not limited to p53, p21, and PUMA in tumor xenografts, peaking at 6 hrs post-dose. Change in the levels of protein markers in degrader treated xenografts was quantified relative vehicle treated xenografts using western blot assay. Clinical equivalent doses of SMIs had no significant in vivo impact in these xenograft models.

Using the mouse xenograft models, an intermittent dosing schedule that drives anti-tumor efficacy is demonstrated. Importantly, a single dose of Compound A at 1 mg/kg resulted in sustained tumor regression in the RS4;11 mouse xenograft model. In this model, Compound A exposures correlated with induction of apoptotic p53 target genes and tumor growth inhibition. In addition, weekly administration of Compound A significantly prolonged the survival in a disseminated AML model when compared to vehicle treated animals.

In summary of FIGS. 1-6, Compound A displays significantly improved potency relative to reversible SMIs leading to potent in vitro and in vivo efficacy that is superior to all clinically active agents. For example, the all cell killing CTG IC₅₀ in RS4;11 for Compound A is 0.3 nm; for DS-3032 (Sankyo/Rain) is 67 nm; for RG7388 (Roche) is 220 nm; for SAR405838 (Sanofi) is 620 nm; for HDM201 (Novartis) is 163 nm; and for AMG-232 (Amgen/Kartos) is 280 nm. In addition, an intermittent dosing schedule of Compound A can induce rapid apoptosis in MDM2-dependent cancer cells potentially leading to improved efficacy and safety profile.

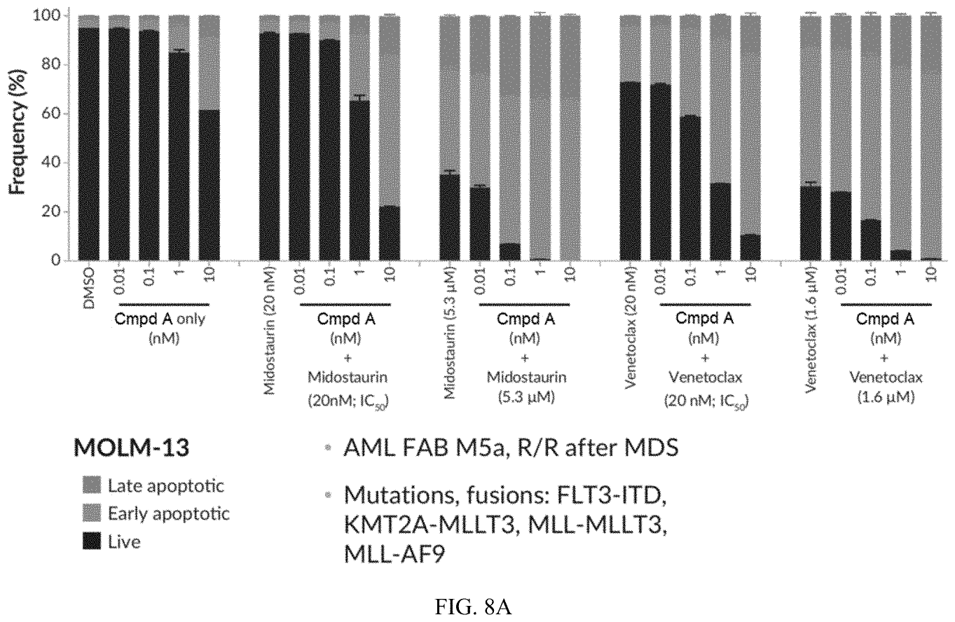

FIGS. 7A and 7B shows that Compound A (1 mg/kg, Q3W) achieves tumor regression in a CTG-2227 AML patient-derived xenograft (PDX) model and partial responses in CTG-2240 and CTG-2700 AML PDX models. Compound A significantly reduces hCD45+ cells in bone marrow and AML blasts.

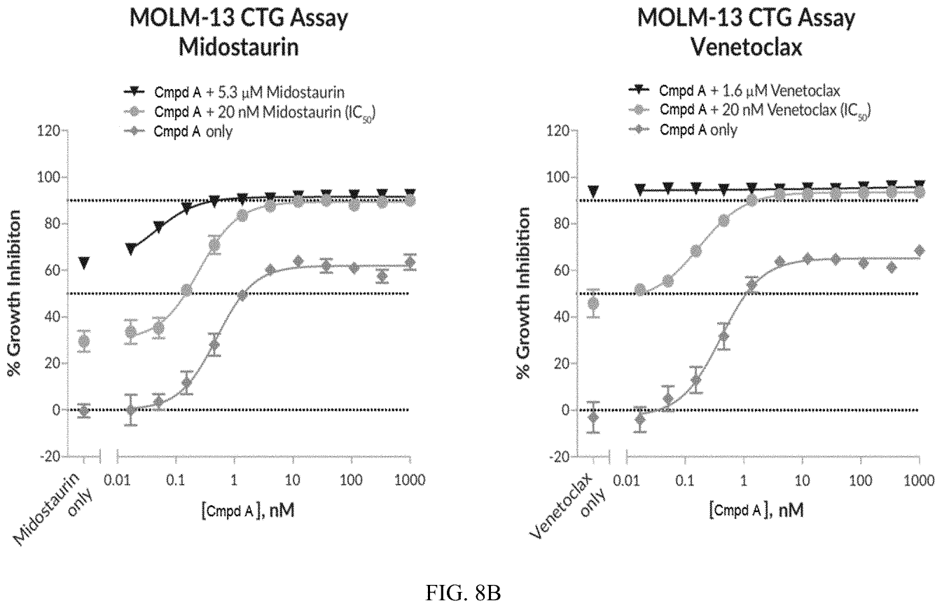

FIGS. 8A and 8B shows the combinatorial benefit of Compound A with venetoclax and midostaurin in MOLM-13 cell line. The data shows that Compound A combination with venetoclax and midostaurin enhances induction of apoptosis and cell killing in MOLM-12 AML cell line.

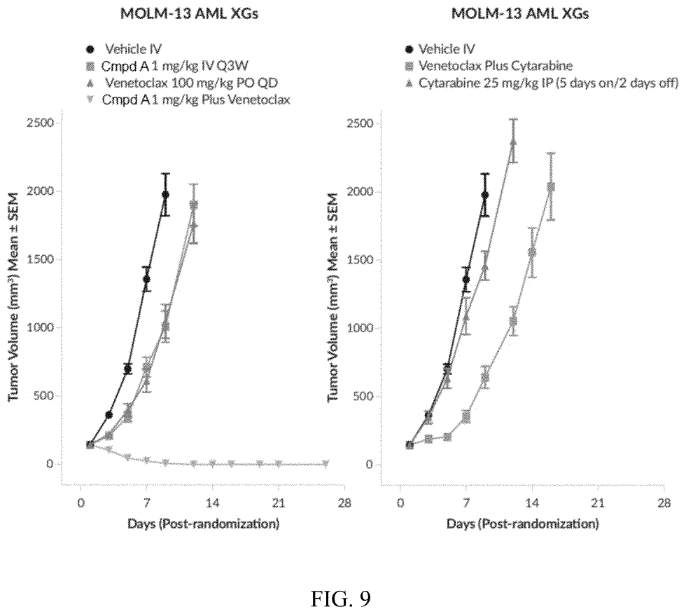

FIG. 9 shows the significant combinatorial benefit of Compound A with standard of care in AML in vivo model. Single dose of Compound A in combination with daily dosing of venetoclax achieves sustained tumor regression in MOLM-13 xenograft model whereas cytarabine or combination of cytarabine and venetoclax demonstrates no significant anti-tumor activity.

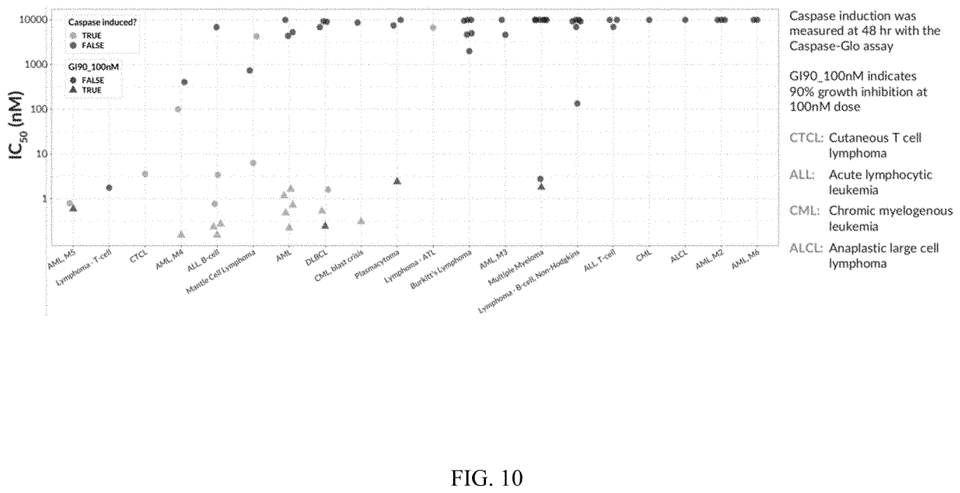

FIG. 10 shows that Compound A is active across multiple heme indications in vitro with AML, T cell lymphomas, mantle cell lymphoma, and DLBCL being the most sensitive.

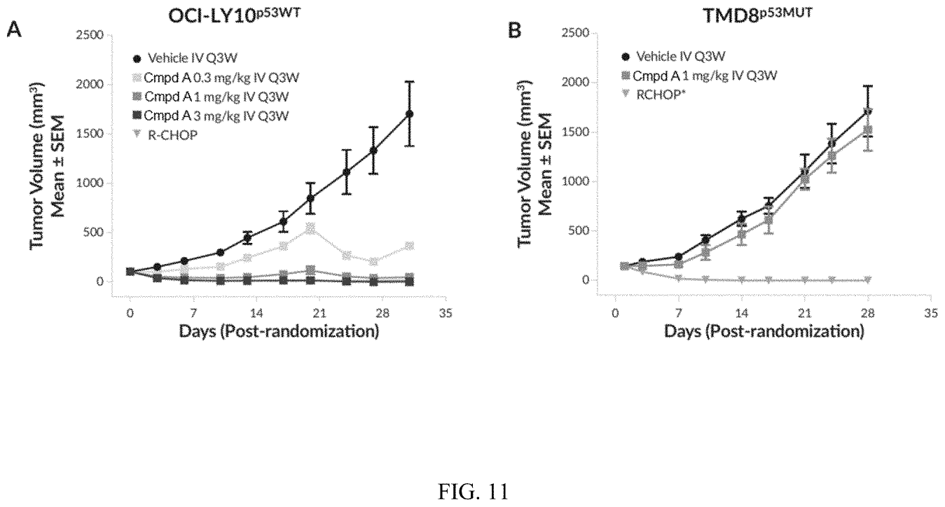

FIG. 11 shows that Compound A is highly active in p53^{WT} ABC-subtype DLBCL. Compound A was highly active in OCI-LY10 p53^{WT} ABC-subtype DLBCL xenograftmodel (A) but not TMD8 p53^{MUT} ABC-subtype DLBCL xenograft model (B).

In summary of FIGS. 7-11, Compound A dosed intermittently is highly active resulting in responses and complete regression in AML PDX xenograft models. Compound A shows combinatorial benefit with SoC agents in AML in-vitro and in-vivo model, suggesting that Compound A combination can be used for larger patient population. Preclinical data suggest potential for Compound A to be active in additional hematological malignancies, such as DLBCL.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating a solid cancer or hematological malignancy in a patient in need thereof, comprising administering a therapeutically effective amount of an MDM2 degrader or a pharmaceutically acceptable salt thereof to the patient; wherein the MDM2 degrader is:
(3′R,4'S,5′R)-6″-chloro-4′-(3-chloro-2-fluorophenyl)-N-((1R,4R)-4-(4-(1-(2,6-dioxopiperidin-3-yl)-3-methyl-2-oxo-2,3-dihydro-1H-benzo[d]imidazol-5-yl)piperidine-1-carbonyl)cyclohexyl)-2″-oxodispiro[cyclohexane-1,2′-pyrrolidine-3′,3″-indoline]-5′-carboxamide (Compound A),
(3′R,4'S,5′R)-6″-chloro-4′-(2-chloro-3-fluoropyridin-4-yl)-N-((6S)-6-((5-(1-(2,6-dioxopiperidin-3-yl)-3-methyl-2-oxo-2,3-dihydro-1H-benzo[d]imidazol-5-yl)pentyl)carbamoyl)tetrahydro-2H-pyran-3-yl)-4,4-dimethyl-2″-oxodispiro[cyclohexane-1,2′-pyrrolidine-3′,3″-indoline]-5′-carboxamide (Compound B),
(3′R,4'S,5′R)-6″-chloro-4′-(3-chloro-2-fluorophenyl)-N-((1r,4R)-4-((2-(2-(1-(2,6-dioxopiperidin-3-yl)-3-methyl-2-oxo-2,3-dihydro-1H-benzo[d]imidazol-5-yl)ethyl)-2,7-diazaspiro[3.5]nonan-7-yl)methyl)cyclohexyl)-1′-methyl-2″-oxodispiro[cyclohexane-1,2′-pyrrolidine-3′,3″-indoline]-5′-carboxamide (Compound C),
(3′R,4'S,5′R)-6″-chloro-4′-(3-chloro-2-fluorophenyl)-N-(4-(4-(1-(2,6-dioxopiperidin-3-yl)-3-methyl-2-oxo-2,3-dihydro-1H-benzo[d]imidazol-5-yl)piperidine-1-carbonyl)phenyl)-2″-oxodispiro[cyclohexane-1,2′-pyrrolidine-3′,3″-indoline]-5′-carboxamide (Compound D), or
(3′R,4'S,5′R)-6″-chloro-4′-(3-chloro-2-fluorophenyl)-N-((1r,4R)-4-(4-((2-(2,6-dioxopiperidin-3-yl)-1-oxoisoindolin-4-yl)ethynyl)piperidine-1-carbonyl)cyclohexyl)-2″-oxodispiro[cyclohexane-1,2′-pyrrolidine-3′,3″-indoline]-5′-carboxamide (Compound E).

2. The method of claim 1, wherein the MDM2 degrader or a pharmaceutically acceptable salt thereof is administered at a dose of up to 0.8 mg/kg to the patient.

3. The method of claim 1, wherein the MDM2 degrader or a pharmaceutically acceptable salt thereof is administered at a dose of up to 0.65 mg/kg to the patient.

4. (canceled)

5. The method of claim 1, wherein the MDM2 degrader or a pharmaceutically acceptable salt thereof is administered at a dose of from about 0.3 mg/kg to about 0.6 mg/kg to the patient.

6. The method of claim 1, wherein the MDM2 degrader or a pharmaceutically acceptable salt thereof is administered at a dose of from about 0.5 mg/kg to about 0.8 mg/kg to the patient.

7-9. (canceled)

10. The method of claim 1, wherein the MDM2 degrader or a pharmaceutically acceptable salt thereof is administered at a dose of from about 10 mg to about 40 mg to the patient.

11. The method of claim 1, wherein the MDM2 degrader or a pharmaceutically acceptable salt thereof is administered at a dose of from about 20 mg to about 50 mg to the patient.

12-16. (canceled)

17. The method of claim 1, wherein the MDM2 degrader or a pharmaceutically acceptable salt thereof is administered orally to the patient.

18. The method of claim 17, wherein the oral administration of the MDM2 degrader to the patient comprises solutions, suspensions, emulsions, tablets, pills, capsules, powders, or sustained-release formulations.

19. The method of claim 1, wherein the MDM2 degrader or a pharmaceutically acceptable salt thereof is administered intravenously to the patient.

20. The method of claim 19, wherein the intravenous administration of the MDM2 degrader to the patient comprises sterile injectable solutions.

21. The method of claim 1, wherein the MDM2 degrader or a pharmaceutically acceptable salt thereof is administered to the patient once weekly (QW).

22. The method of claim 1, wherein the MDM2 degrader or a pharmaceutically acceptable salt thereof is administered to the patient once every two weeks (Q2W).

23. The method of claim 1, wherein the MDM2 degrader or a pharmaceutically acceptable salt thereof is administered to the patient once every three weeks (Q3W).

24. The method of claim 1, wherein the MDM2 degrader or a pharmaceutically acceptable salt thereof is administered as a pharmaceutical composition comprising one or more pharmaceutically acceptable excipient or carrier.

25. The pharmaceutical composition of claim 24, wherein the one or more pharmaceutically acceptable excipient or carrier comprises one or more diluents, preservatives, binders, lubricants, disintegrators, swelling agents, fillers, or stabilizers.

26. The pharmaceutical composition of claim 24, wherein the one or more pharmaceutically acceptable excipient or carrier comprises one or more buffers, surfactants, dispersants, emulsifiers, or viscosity modifying agents.

27. The method of claim 1, wherein the solid cancer or hematological malignancy is selected from acute lymphoblastic leukemia (ALL), chronic lymphocytic leukemia (CLL), large granular lymphocytic leukemia (LGL-L), B-cell prolymphocytic leukemia, acute myeloid leukemia (AML), Burkitt lymphoma/leukemia, primary effusion lymphoma, peripheral T-cell lymphoma (PTCL), cutaneous T-cell lymphoma (CTCL), diffuse large B-cell lymphoma (DLBCL), advanced B-cell diffuse large B-cell lymphoma (ABC DLBCL), intravascular large B-cell lymphoma, lymphoplasmacytic lymphoma, Waldenström's macroglobulinemia (WM), splenic marginal zone lymphoma, multiple myeloma, plasmacytoma, uveal melanoma, or myelodysplastic syndrome (MDS).

28. The method of claim 1, wherein the patient has received at least one prior therapy.

29. The method of claim 1, wherein the patient is a human.
